(19) United States
(12) Patent Application Publication
(10) Pub. No.: US 2024/0242784 A1
(43) Pub. Date: Jul. 18, 2024

(54) MOLECULAR ENERGY PREDICTION METHOD AND APPARATUS, DEVICE, AND STORAGE MEDIUM

(71) Applicant: Tencent Technology (Shenzhen) Company Limited, Shenzhen (CN)

(72) Inventors: Lixue CHENG, Shenzhen (CN); Jonathan Pradana MAILOA, Shenzhen (CN); Shengyu ZHANG, Shenzhen (CN)

(21) Appl. No.: 18/619,052

(22) Filed: Mar. 27, 2024

Related U.S. Application Data
(63) Continuation of application No. PCT/CN2023/103429, filed on Jun. 28, 2023.

(30) Foreign Application Priority Data
Oct. 18, 2022 (CN) 202211274957.6

(51) Int. Cl. G16C 20/10; G16C 20/70

(52) U.S. Cl. CPC G16C 20/10; G16C 20/70

(57) ABSTRACT

This application discloses a molecular energy prediction method performed by a computer device. The method includes: obtaining first prediction energy of a target molecule and a quantum operator of the target molecule by using a first calculation method, the quantum operator of the target molecule being configured for describing a wave function of the target molecule; predicting energy information of the target molecule through a molecular energy prediction model and according to the quantum operator of the target molecule; and determining final prediction energy of the target molecule according to the first prediction energy and the energy information. The first prediction energy of the target molecule and the quantum operator of the target molecule obtained through the first calculation method are used to predict the final prediction energy of the target molecule according to the molecular energy prediction model.

### CROSS-REFERENCE TO RELATED APPLICATIONS

This application is a continuation application of PCT Patent Application No. PCT/CN2023/103429, entitled “MOLECULAR ENERGY PREDICTION METHOD AND APPARATUS, DEVICE, AND STORAGE MEDIUM” filed on Jun. 28, 2023, which claims priority to Chinese Patent Application No. 202211274957.6, entitled “MOLECULAR ENERGY PREDICTION METHOD AND APPARATUS, DEVICE, AND STORAGE MEDIUM” filed on Oct. 18, 2022, all of which is incorporated herein by reference in its entirety.

### FIELD OF THE TECHNOLOGY

Embodiments of this application relate to the field of quantum technologies, and in particular, to a molecular energy prediction method and apparatus, a device, and a storage medium.

### BACKGROUND OF THE DISCLOSURE

In quantum chemistry, molecular energy is predicted to calculate a molecular reaction mechanism, calculate a molecular spectra, and the like. Therefore, prediction of the molecular energy has far-reaching practical significance.

In the related art, the molecular energy is predicted through molecular structural information. Generally, the molecular structural information, such as a bond type, a bond length, a bond angle, and other information, is used as an input of a molecular energy prediction model, and the molecular energy is predicted by the model.

However, in the related art, the molecular energy is predicted according to the molecular structural information. Since each molecule has a large amount of structural information, and structures between different molecules are inconsistent, causing high calculation costs and poor transferability.

### SUMMARY

Embodiments of this application provide a molecular energy prediction method and apparatus, a device, and a storage medium. The technical solutions are as follows:

According to an aspect of the embodiments of this application, a molecular energy prediction method is provided, and is performed by a computer device. The method includes:
obtaining first prediction energy of a target molecule and a quantum operator of the target molecule by using a first calculation method, the quantum operator of the target molecule being configured for describing a wave function of the target molecule;
predicting energy information of the target molecule through a molecular energy prediction model and according to the quantum operator of the target molecule; and
determining final prediction energy of the target molecule according to the first prediction energy and the energy information.

According to an aspect of the embodiments of this application, a computer device is provided, including a processor and a memory, the memory storing a computer program, the computer program being loaded and executed by the processor to implement the foregoing molecular energy prediction method.

According to an aspect of the embodiments of this application, a non-transitory computer-readable storage medium is provided, storing a computer program, the computer program being loaded and executed by a processor of a computer device to implement the foregoing molecular energy prediction method.

The technical solutions provided in the embodiments of this application may include the following beneficial effects: The first prediction energy of the target molecule and the quantum operator of the target molecule are obtained through the first calculation method (a calculation method at a lower cost). By inputting the quantum operator into the molecular energy prediction model, the energy information about the target molecule may be obtained. Through the energy information and the first prediction energy, the final prediction energy of the target molecule may be determined. The final prediction energy of the target molecule is more accurate than the first prediction energy. That is, the technical solutions provided in the embodiments of this application take the quantum operator of the molecule as an input and predicts energy of the molecule through the molecular energy prediction model. Since there are not many types of quantum operators, the types of quantum operators are basically the same between different molecules, so the molecular energy prediction model has good transferability, and the molecular energy prediction method has good universality. In addition, since the first prediction energy is obtained through a molecular energy calculation method at the lower calculation cost, the technical solutions provided in the embodiments of this application can predict the molecular energy with higher precision at the lower calculation cost.

### BRIEF DESCRIPTION OF THE DRAWINGS

FIG. 1 is a schematic diagram of a coordinate relationship between a calculation cost provided in the related art and an exact numerical solution of a Schrödinger equation of a corresponding system.

FIG. 2 is a schematic diagram of an application manner of machine learning provided in the related art in various subsidiary fields of computational chemistry.

FIG. 3 is a schematic diagram of predicting a molecular energy using a machine learning method according to an embodiment of this application.

FIG. 4 is a schematic diagram of calculating a calculation cost required by a catalyst using different methods according to an embodiment of this application.

FIG. 5 is a schematic diagram of a potential energy surface in an actual simple reaction according to an embodiment of this application.

FIG. 6 is a schematic diagram of a solution implementation environment according to an embodiment of this application.

FIG. 7 is a flowchart of a molecular energy prediction method according to an embodiment of this application.

FIG. 8 is a block diagram of a method for obtaining operator information according to an embodiment of this application.

FIG. 9 is a block diagram of a molecular energy prediction method according to an embodiment of this application.

FIG. 10 is a flowchart of a method for training a molecular energy prediction model according to an embodiment of this application.

FIG. 11 is a schematic diagram of a prediction result of an electronic structural energy according to an embodiment of this application.

FIG. 12 is a schematic diagram of a prediction result of a multi-molecule standardized dataset according to an embodiment of this application.

FIG. 13 is a block diagram of a molecular energy prediction apparatus according to an embodiment of this application.

FIG. 14 is a block diagram of a molecular energy prediction apparatus according to another embodiment of this application.

FIG. 15 is a block diagram of an apparatus for training a molecular energy prediction model according to an embodiment of this application.

FIG. 16 is a block diagram of an apparatus for training a molecular energy prediction model according to another embodiment of this application.

FIG. 17 is a block diagram of a structure of a computer device according to an embodiment of this application.

### DESCRIPTION OF EMBODIMENTS

To make objectives, technical solutions, and advantages of this application clearer, implementations of this application are further described below in detail with reference to the accompanying drawings.

Before technical solutions of this application are described, some terms involved in this application are first explained and described. The following relevant explanations may be arbitrarily combined with the technical solutions of embodiments of this application as optional solutions, and they all fall within the protection scope of the embodiments of this application. The embodiments of this application include at least some of the following content.

Quantum simulation: It creates a quantum computer similar to or related to a quantum problem to be studied for simulation (natural evolution in an artificially established quantum operating environment).

Quantum computing: It resolves a specific problem, and algorithms used are coherent and reversible operations.

Operator: It is a function from a physical state space to another physical state space. This application mainly uses operators that can describe wave functions in quantum chemical calculations, including a single-electron operator and a two-electron operator. For example, a Fock operator (represented as a matrix) is a single-electron energy operator (matrix) that approximates a given quantum system in a given set of basis vectors.

Schrödinger equation (SE): It is a partial differential equation that describes evolution of a quantum state of a physical system over time, and is one of basic equations of quantum mechanics.

Electronic structure: It is a scientific research method and field that solves the Schrödinger equation by solving a wave function of electrons through Born-Oppenheimer approximation.

Wave function theory (WFT): It is a quantum mechanical method based on an electronic structure of a multi-electron system based on a complex multi-electron wave function.

Density functional theory (DFT): It is a quantum mechanical method that uses electron density to study an electronic structure of a multi-electron system, and its main objective is to use the electron density to replace a wave function as a basic quantity of research.

Weakly-correlated and strongly-correlated: They describe strength of interaction between electrons in a system. It is generally considered that a quantum simulation method with low accuracy can also handle a weakly-correlated system, but a strongly-correlated system requires an electronic structure theory method with high-accuracy based on a wave function for handling.

Self-consistent field method (SCF): It is a basic method for iteratively solving the Schrödinger equation of a multi-particle system in quantum mechanics. In the embodiments of this application, the particle specifically refers to an electron. The self-consistent field method first gives an estimate of a wave function to estimate electron density, then uses the electron density to obtain terms related to interaction between particles in the Hamiltonian, and then solves the Schrödinger equation to obtain a set of improved estimates. There are many self-consistent field methods that can be selected in the technical solutions provided in the embodiments of this application, such as Hartree-Fock (HF), a Hartree method, a multi-configuration self-consistent field method, and the like

Ground state and excited state: The ground state is a quantum state with the least energy among a series of quantum states possessed by a system, and the excited state is a series of quantum states other than the ground state in a system.

Gaussian process: It is a random process in which observations occur in a continuous domain (time or space). In the Gaussian process, each point continuously inputted in a space is associated with a normally distributed random variable, and any finite linear combination of random variables is a normal distribution.

Gaussian process regression: It is a non-parametric model that uses the Gaussian process prior to perform regression analysis on data, and is also a probabilistic model that is versatile and analytic.

Addition kernel function, kernel matrix, and kernel-addition Gaussian process regression (KA-GPR): Assuming that each small cell conforms to a uniform Gaussian process, a sum of these small cells is also a Gaussian process (referred to as a kernel-addition Gaussian process), and a kernel function of the Gaussian process is an addition kernel function, and a matrix obtained by inputting representation information to the kernel function is represented as a kernel matrix.

Before the technical solutions of this application are described, some relevant background knowledge related to this application is first explained and described.

### 1. Electronic Structural Method in Related Quantum Simulation

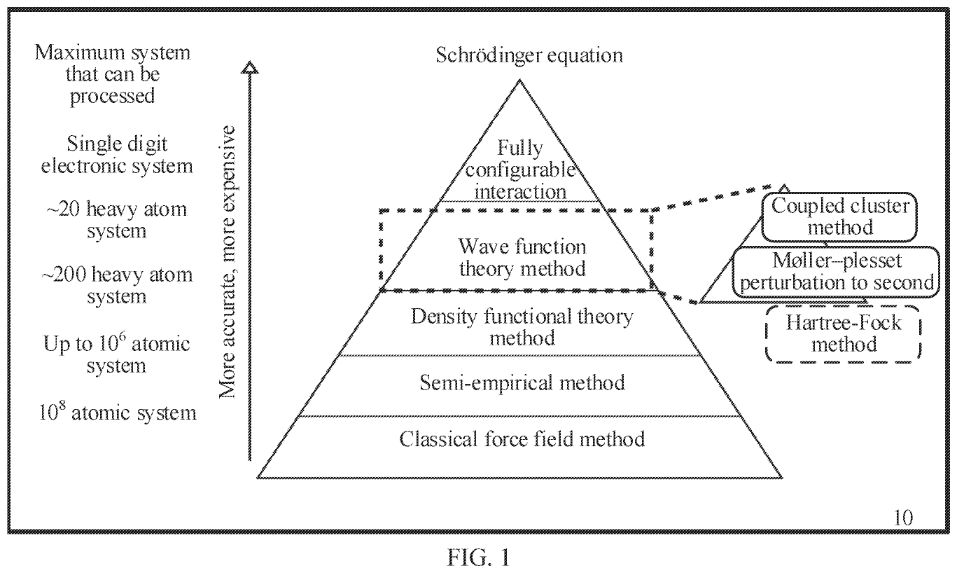

As a powerful and widely used calculation tool, quantum simulation has been shown to be capable of deepening the understanding of chemical and biological processes, and facilitating the discovery of new drugs and new materials. An ultimate objective of the quantum simulation is to find an exact numerical solution to the Schrödinger equation of a corresponding system with a reasonable calculation cost. As shown in a coordinate system 10 shown in FIG. 1, which shows a commonly used method of solving the Schrödinger equation in computational chemistry and a maximum system that can be calculated, it can be found that the calculation cost and the calculation complexity increase as the precision of the method increases, while the maximum system that can be processed also decreases significantly. FIG. 1 is a pyramid of solving the Schrödinger equation by using the commonly used method in the computational chemistry. In the field of electronic structures, trade-offs of cost and precision of various theory calculation methods developed by physics and chemists make it difficult to compromise both in an actual system calculation. While the advent of the density functional theory (DFT) partially solves the problem that the electronic structure calculation cannot be performed in an actual system, but the calculation accuracy of the DFT with respect to the energy is difficult to achieve actual requirements of some application problems. The wave function theory is generally considered to provide a more accurate solution to the Schrödinger equation, but a more practical method is the Kohn-Sham density functional theory. The advent of the density functional theory allows a traditional electronic structural method to deal with an actual chemical biological system. However, in many applications, the density functional theory has many quantitative and even qualitative errors, so how to quickly obtain a nearly accurate numerical solution to the wave function theory method and even complete configuration interaction method is an important issue in the field of electronic structural research.

### 2. Machine Learning in the Field of Computational Chemistry

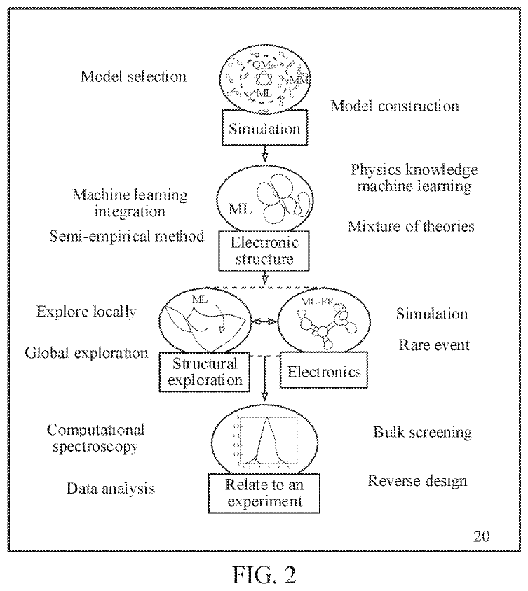

As machine learning gradually exhibits strong calculation efficiency in various industries, to give consideration to both accuracy and the calculation cost, the field of computational chemistry also begins to introduce a machine learning method on a large scale for industrial upgrades and innovations. FIG. 2 shows various ways of application of machine learning in various affiliated fields of computational chemistry, and the machine learning may be applied to various fields 20 shown in FIG. 2. The interworking of these affiliated fields 20 further facilitates the integration of overall computational chemistry with the machine learning. There are various different ways to apply the machine learning for the specialized field of electronic structures. In the related art, there are two main categories of machine learning methods applied in the field of electronic structures and molecular energy learning, namely: machine learning based on molecular structural information and machine learning based on quantum mechanical information.

### 2.1 Machine Learning Based on Molecular Structural Information

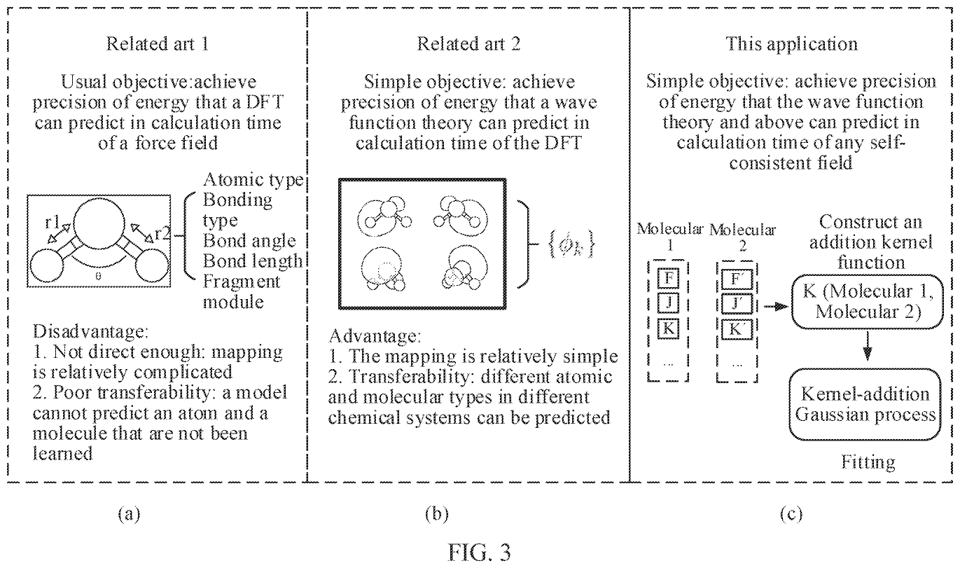

The first category of machine learning methods based on molecular structural information focus on being able to achieve excellent precision in predicting molecular energy at a DFT level by using the calculation cost of a classical force field. These methods generally use the molecular structural information to describe chemical systems, such as atomic composition, bonding types, bond lengths, and bond angles. As shown in FIG. 3(a), which shows replacement of a more expensive electronic structure potential energy surface and facilitating detailed introduction to molecular dynamics simulations in large chemical systems over 100000 atoms with precision achievable by DFT. However, this category of machine learning method represented by molecular structural information has two notable drawbacks. First, as quantities of atoms and bond types increase, a quantity of features grows rapidly, and the complexity of constructing a machine learning model that can accurately describe different elements and chemicals grows quickly. In addition, there is a significant loss of precision in the prediction of untrained elements and chemical environment types due to the lack of relevant information. These two problems result in that in training, this category of machine learning methods represented by molecular structural information inevitably require large amounts of reference data (generally more than 50000 training molecules) to achieve the precision required for chemical applications, and lack transferability in different chemical problems.

### 2.2 Machine Learning Based on Quantum Mechanical Information

An objective of the second category of machine learning methods based on quantum mechanical information is to achieve precision on the wave function using information from a low-level electronic structural theory. As shown in FIG. 3(b), chemical systems are usually described using physical information representation (or quantum representation) obtained from quantum simulation calculation, where the physical information representation usually chosen is molecular or atomic orbital information. Quantum information used in this category of machine learning includes an atomic orbital, a molecular orbital, and a Slater determinant obtained from Hartree-Fock (HF) or DFT. Compared with the machine learning method represented by the molecular structural information, the machine learning method using molecular or atomic orbital information generally requires fewer data points (generally less than 5000) than the machine learning method using the molecular structural information for representation to achieve the same precision. In addition, the machine learning methods based on molecular or atomic orbital information may further achieve better model mobility, and such methods also generally outperform the method based on molecular structural information on large standard datasets. The machine learning methods based on molecular or atomic orbital information currently have many options, such as NeuralXC, DeePHF, DeePKS, PauliNet, and OrbNet.

### 3. Shortcomings in the Related Art

The following problems still exist in the related art:

### 3.1 Small Data Model and Big Data Model

Although a small data model can obtain very high precision for individual application scenarios and even individual specific chemical systems, it lacks ubiquity and good mobility. Although a big data model has a good prediction capability for different architectures and scenarios, it does not have a capability to perform model update iterations for individual applications. Although there are methods that have the potential to be universally suited to the small data model and the big data model, there is a need to rely on deep machine learning algorithm development. Taking the molecular orbital based machine learning (MOB-ML) method as an example, the most straightforward MOB-ML method uses traditional Gaussian process regression, which requires recalculation of a kernel matrix every cycle in the process of optimizing parameters if not relying on deep development on machine learning. A bottleneck is to invert the kernel matrix (complexity of O(N3), where N is a quantity of training data), and only up to 200 molecules can be trained due to a special training design that decomposes the total energy. Various additional machine learning techniques such as clustering and approximation enable MOB-ML to train the big data model.

### 3.2 Single Input and Output, Lack of Versatility and Cannot Adapt to Many Application Fields

Most models are developed towards specific electronic structural theory, with fixed input and output target theories. For example, an input end of a model is a result of semi-empirical theory calculation and an output end is a prediction value of DFT theory calculation result. Since there are different requirements on precision and target theories for different chemical systems and application scenarios, a user using the model needs to determine in advance whether the model provides the output theory precision that is consistent with the system and the application that the user intends to study. In particular, most methods lack a modelling capability towards the electronic structural theory with high precision, resulting in an inability to adapt to many application scenarios. The objective of most current machine learning models is to achieve DFT level precision. For many application scenarios, such as a molecular system that is strongly-correlated or in the excited state, a corresponding chemical system can be accurately described only by providing a higher-precision quantum simulation calculation result. However, almost only energy of a weakly-correlated molecular system in the ground state can be predicted in the related art.

### 3.3 Lack of Transferability of Small Molecule System Model to Macromolecule System

Most models lack transferability and predictability across molecular sizes. Generally, most methods may obtain machine learning models with very high precision for a particular molecular size dataset, but these models generally suffer from a large loss of precision when a larger molecular system is predicted.

4. Advantages of the Technical Solutions Provided in the Embodiments of this Application

The technical solutions provided in the embodiments of this application provide an efficient and universal quantum representation-based machine learning method (belonging to the second category of methods), as shown in FIG. 3(c), which may be referred to as a quantum operator-based machine learning (OBML) method. This method provides an efficient, accurate, universal, transferable, and universal molecular energy prediction method by using the matrix of a quantum operator and its possible matrix operation result as input information and the kernel-addition Gaussian process as a machine learning fitting algorithm. The technical solutions provided in the embodiments of this application have three significant features as follows:

### 4.1 Compatibility: Adapting a Small Data Customized Model and a Big Data Universal Model

The technical solutions provided in the embodiments of this application are currently to use Gaussian process as a machine learning algorithm. The Gaussian process is an extremely accurate machine learning method, and generally requires little data to achieve relatively high precision compared to a neural network, which gives the user a possibility of using a small amount of data for targeted local modeling. With respect to the big data model part, although OBML is a new technology and the machine learning framework is currently based on traditional Gaussian process regression, it has a capability to learn big data.

### 4.2 Pervasive: Utilizable Inputs and Outputs of a Plurality of Target Accuracies, and Adapting to a Wider Range of Application Scenarios

The technical solutions provided in the embodiments of this application can support any reasonable self-consistent field theory calculation information as an input, and can obtain an OBML model with a corresponding precision as long as data of a reasonable ground state high-precision wave function theory is trained, and there is no strict requirement for the input end theory and the output end theory. The technical solutions provided in the embodiments of this application can predict a high-precision quantum simulation theory result, and can also select a suitable input and output theory for problems in different chemical fields, thereby adapting to a wider application scenario.

### 4.3 Transferability: Instead of Directly Including Training Data for a Macromolecular System, a Small Molecular System Model can Also Accurately Predict the Molecular Energy for the Macromolecular System.

The embodiments of this application can be used to improve the calculation efficiency of many traditional quantum chemical simulation traditional problems, and can also provide energy prediction for a system that some traditional quantum simulation calculation methods cannot calculate. These traditional problems include high-precision single-molecule ground-state energy calculation, provision of a high-precision potential energy plane for high-efficient molecular dynamics simulation, and construction of a multi-molecule universal molecular energy prediction model.

### 1. High-Precision Single-Molecule Ground-State Energy Calculation

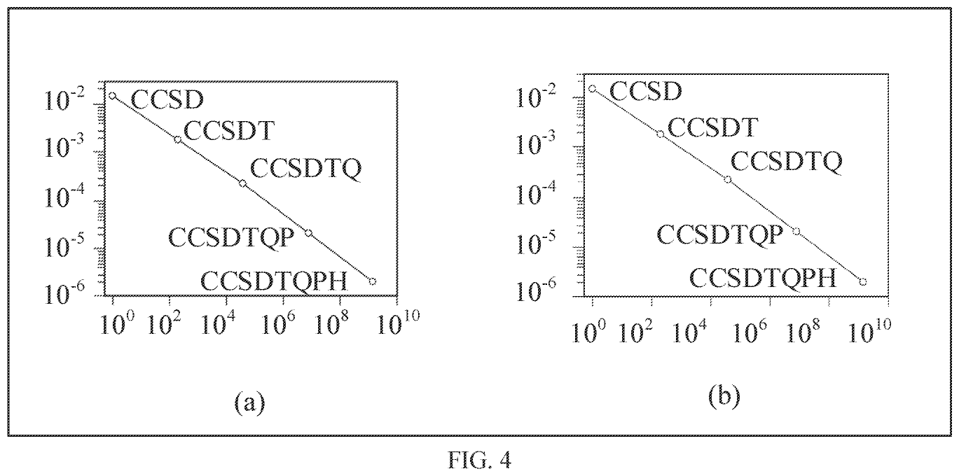

Strongly-correlated phenomena exist in many chemical systems of practical value, such as a metal organic catalyst, a material, a superconductor, and the like. However, theoretical chemical calculation of a strongly-correlated system is very difficult. First, the calculation of the strongly-correlated system requires high precision. Since most of the strongly-correlated systems with application value require high-precision theory calculation while the systems are large, a practical system cannot be calculated without any approximation. FIG. 4 shows the calculation cost required to calculate a catalyst using different accurate wave function methods and using an approximation algorithm in a small system. FIG. 4(a) shows time (in seconds) required for various high-precision wave function methods to calculate an N2 molecule. The five methods listed are all coupled cluster methods, and the higher the quantity of excitations considered the more accurate the energy of the molecule the method predicts, S (singles), D (doubles), T (triples), Q (quadraples), P (pentaples), and H (Hexaples). FIG. 4(b) shows time required to calculate a small part of an optical system II by using a low-complexity approximation algorithm. OBML requires only very inexpensive self-consistent field theory as an input to achieve the same precision as the accurate wave function method, and can obtain a model that is equally applicable to the large system by training a small system with similar properties. In this way, OBML can be used to achieve calculation acceleration of more than 1000 times, and make calculation that cannot be achieved by traditional methods possible.

### 2. High-Precision Potential Energy Plane

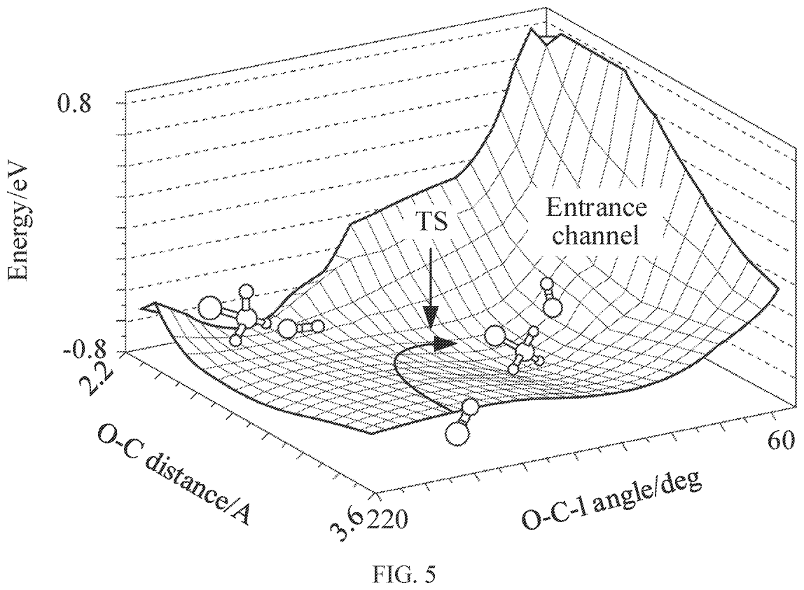

FIG. 5 shows a potential energy plane in an actual simple reaction. In quantum simulation, molecular dynamics is a very good tool for studying reaction mechanisms and processes. However, since molecular dynamics requires the calculation of millions of single-point system energy in the process, the energy calculation used in the molecular dynamics usually cannot achieve the high precision within a reasonable time calculation cost. In addition, since shapes of these potential energy planes are too complex, simple functional fitting usually does not work well, or requires a lot of reference calculations. Since OBML can use semi-empirical self-consistent field theory as input information, OBML calculates energy at a rate close to the potential plane used by the traditional molecular dynamics. However, OBML can provide more accurate energy, thereby improving the precision of the overall molecular dynamics simulation, ultimately achieving a more accurate description of the overall reaction mechanism.

### 3. Multi-Molecule Universal Molecular Energy Prediction Model

A universal molecular property prediction model is always a very popular direction in the field of machine learning electronic structures. By training a variety of different molecules simultaneously rather than training only different configurations of the same molecule, a universal molecular energy prediction model can be constructed. By training molecular energy data of different wave function theories, a molecular energy prediction model with different wave function theories as target precision can be constructed. Such a multi-molecule universal molecular energy prediction model can broadly predict a variety of different molecular energy under a variety of different scenarios.

Therefore, the technical solutions provided in the embodiments of this application provide an efficient, accurate, and transferable molecular energy model construction strategy for assisting quantum chemical simulation calculation using the machine learning method. By using various quantum operators describing the properties of single electrons and double electrons and related operator operations provided by the low-precision self-consistent field method as input information, in combination with kernel-addition Gaussian process regression algorithms, the energy data of the high-precision wave function method is trained to obtain an exact and physically meaningful high-precision molecular energy model. The technical solutions provided in the embodiments of this application aim to improve the calculation capability and precision of machine learning-based calculation quantum chemistry to a new level, at a cost significantly lower than the traditional quantum simulation. In the technical solutions provided in the embodiments of this application, benchmark databases of a variety of applications are tested for the common scenario of ground state energy of the molecular system, and are systematically compared with other state-of-the-art machine learning solutions to illustrate advantages of the technical solutions provided in the embodiments of this application in terms of calculation time and precision.

The technical solutions provided in the embodiments of this application are applied to the field of quantum chemistry. In addition, the technical solutions provided in this application can be applied to energy prediction of any molecule, that is, the mentioned molecules in the technical solutions provided in the embodiments of this application may be any one or more existing molecules or any one or more new molecules discovered in the future, and a specific molecule name or molecule type is not limited in this application. In some embodiments, the molecule may be a molecule in the ground state (that is, atoms that make up the molecule are atoms in the ground state) or a molecule in the excited state (that is, atoms that make up the molecule are atoms in the excited state). In some embodiments, the molecule may be a macromolecule or a high polymer, or may be a small molecule. For example, the molecule includes, but is not limited to, a water molecule, a carbon dioxide molecule, a hydrogen gas molecule, and the like.

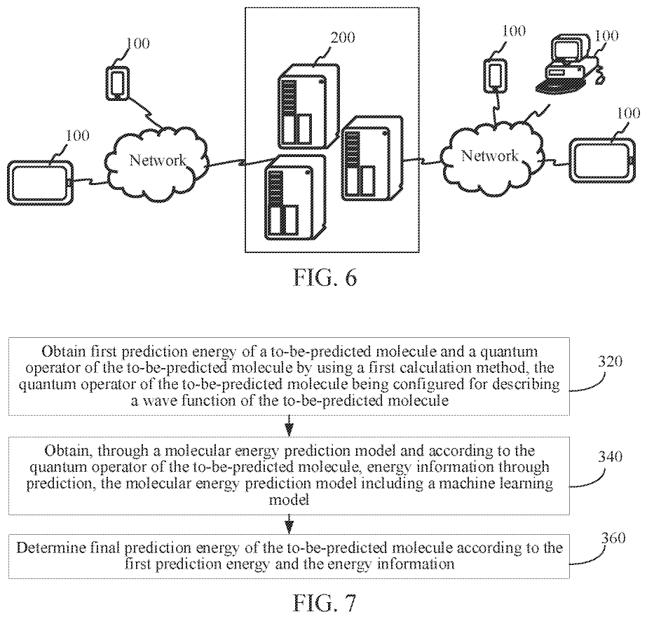

Referring to FIG. 6, which shows a schematic diagram of a solution implementation environment according to an embodiment of this application. The solution implementation environment may include: a terminal device 100 and a server 200.

The terminal device 100 includes, but is not limited to, an electronic device such as a mobile phone, a tablet computer, an intelligent voice interactive device, a game host, a wearable device, a multimedia playing device, a personal computer (PC), a vehicle-mounted terminal, a smart appliance, and the like. A client of a target application program may be mounted on the terminal device 100.

In the embodiments of this application, the target application program may be any application program that provides molecular energy prediction, specifically, may be a quantum chemical type application program, a virtual reality (VR) type application program, an augmented reality (AR) type application program, or the like, which is not limited in the embodiments of this application. In some embodiments, the terminal device 100 has a client running the foregoing target application program.

The server 200 is configured to provide a backend service for the client of the target application program in the terminal device 100. The server 200 may be an independent physical server, or may be a server cluster or a distributed system formed by a plurality of physical servers, or may be a cloud server that provides basic cloud computing services such as a cloud service, a cloud database, cloud computing, a cloud function, cloud storage, a network service, cloud communication, a middle ware service, a domain name service, a security service, a content delivery network (CDN), big data, and an artificial intelligence platform, which is not limited to this.

The terminal device 100 and the server 200 may communicate with each other through a network. The network may be a wired network or a wireless network.

The embodiments of this application provide methods in which steps may be performed by a computer device. The computer device may be any electronic device having data storage and processing capabilities. For example, the computer device may be the server 200 in FIG. 6, may be the terminal device 100 in FIG. 6, or may be another device other than the terminal device 100 and the server 200.

Referring to FIG. 7, which is a flowchart of a molecular energy prediction method according to an embodiment of this application. Each step of the method may be performed by the terminal device 100 in the solution implementation environment shown in FIG. 6, or by the server 200 in the solution implementation environment shown in FIG. 6. In the following method embodiment, for ease of description, a computer device is used as an execution body of each step for description. The method may include at least one step in the following steps (320 to 360):

Step 320: Obtain first prediction energy of a target molecule and a quantum operator of the target molecule by using a first calculation method, the quantum operator of the target molecule being configured for describing a wave function of the target molecule.

The first prediction energy is prediction energy of the target molecule obtained by using the first calculation method. The first calculation method may be a self-consistent field theory method. The target molecule may be an electron, a free radical small molecule, a large standard organic compound molecule, and the like.

The basic idea of the self-consistent field theory method is: First an estimate of a wave function is given to estimate electron density, then the electron density is used to obtain terms related to interaction between particles in the Hamiltonian, and then the Schrödinger equation is solved to obtain a set of improved estimates. The set of estimates includes eigenvalues and eigenvectors, where the eigenvalues are eigenvalues of the quantum operator and a minimized eigenvector is the prediction energy of the molecular.

In some embodiments, step 320 includes step 320-2 (not shown in the figure).

Step: 320-2: Obtain the first prediction energy of the target molecule and the quantum operator of the target molecule by using any self-consistent field theory method.

In some embodiments, the self-consistent field theory method may include at least one of the following: a multi-configuration self-consistent field method, a density functional theory, and a HF method.

In some embodiments, loop calculation may be performed according to steps “initialize a quantum state, calculate current state density or an orbital, calculate current energy, obtain new density or a new orbital from gradient update, and calculate new energy” until the gradient on the state is substantially zero and no further state update can be continued. The finally obtained energy is determined as the first prediction energy of the target molecule.

In some embodiments, the first prediction energy of the target molecule may be obtained through the following steps: a first step of estimating a wave function to obtain a linear combination coefficient of a basis function in an estimated molecular orbital; a second step of estimating electron density and calculating gradient; and a third step of obtaining an improved estimate, obtaining eigenvalues and eigenvectors according to the improved estimate, using the improved estimate as estimate of a linear combination coefficient of a new basis function, and returning to the first step. The minimized eigenvector is the first prediction energy and the eigenvalues are eigenvalues of the quantum operator of the target molecule.

In some embodiments, the self-consistent field theory method such as the HF method is used to obtain the first prediction energy of the target molecule and the quantum operator of the target molecule. In a first step, a wave function is estimated to obtain a linear combination coefficient of a basis function in an estimated molecular orbital; in a second step, electron density is estimated and a density matrix is calculated; in a third step, an interaction term is calculated and a Fock matrix element is calculated; and in a fourth step, an improved estimate is obtained, eigenvalues and eigenvectors are obtained by diagonalizing the Fock matrix, the improved estimate is used as estimate of a linear combination coefficient of a new basis function, and the first step is returned. That is to say, the wave function is first estimated to obtain the linear combination coefficient of the basis function in the estimated molecular orbital; further, the electron density is estimated to obtain an estimated electron density, and a density matrix is calculated according to the estimated electron density; a term in the Hamiltonian related to the inter-particle interaction (that is, the foregoing interaction term) according to the density matrix; a Fock matrix element according to the interaction term; and the Schrödinger equation is solved for the Fock matrix element to obtain an improved set of estimates (that is, the obtaining eigenvalues and eigenvectors by diagonalizing the Fock matrix, and using the improved estimate as estimate of a linear combination coefficient of a new basis function). The minimized eigenvector is the first prediction energy and the eigenvalues are eigenvalues of the quantum operator of the target molecule.

The embodiments of this application do not limit the specific form of the first calculation method, and any algorithm provided in the related art that can calculate molecular energy can be considered as the first calculation method in the embodiments of this application.

The embodiments of this application can support any reasonable self-consistent field theory calculation information as an input, and can obtain an OBML model with a corresponding precision as long as data of a reasonable ground state high-precision wave function theory is trained, and there is no strict requirement for the input end theory and the output end theory. The embodiments of this application can predict a high-precision quantum simulation theory result, and can also select a suitable input and output theory for problems in different chemical fields, thereby adapting to a wider application scenario.

In some embodiments, expression forms of the quantum operator include at least one of the following: a structural operator, an atomic orbital operator, and a molecular orbital operator; the structural operator is determined based on a structure of the target molecule; the atomic orbital operator is determined based on an atomic orbital expression form of the target molecule; and the molecular orbital operator is determined based on a molecular orbital expression form of the target molecule. This application also does not limit the specific expression form of the operator.

In some embodiments, types of the quantum operator include at least one of the following: an overlap operator, a kinetic energy operator, a nuclear potential energy operator, a density operator, a Coulomb operator, a commutative operator, and a Fock operator. This application also does not limit the type of the operator.

In the technical solutions provided in the embodiments of this application, molecular characterization is not directly constructed, but construction of the addition kernel function of the molecular characterization is directly attempted. The input end of the kernel function is constructed by using single-electron and two-electron quantum operators under a molecular or atomic orbital basis set. Optional operators include overlap (S), kinetic energy (T), nuclear potential energy (V), density (D), coulomb (J), commutative (K), and Fock (F) operators. For any two electrons p and q of a molecule, a corresponding electron operator is defined as:

$${S}_{pq} = 〈 {ϕ}_{p} \vert {ϕ}_{q} 〉$$

$${T}_{pq} = 〈 {ϕ}_{p} \vert \frac{{\hat{p}}^{2}}{2 m} \vert {ϕ}_{q} 〉$$

$${V}_{pq} = 〈 {ϕ}_{p} \vert - {\sum }_{i} \frac{1}{\vert \hat{r} - {R}_{i} \vert} \vert {ϕ}_{q} 〉$$

$${D}_{pq} = 〈 {Ψ}_{0} \vert {a}_{p} {a}_{q}^{+} \vert {Ψ}_{0} 〉$$

$${J}_{pq} = 〈 pq \vert pq 〉$$

$${K}_{pq} = 〈 pq \vert qp 〉$$

$${F}_{p} = {h}_{p} + {\sum }_{q = 1}^{n / 2} 2 * {J}_{pq} - {K}_{pq} )$$

ϕ is an atomic or molecular orbital, a⁺ and a are a generation operator and an annihilation operator of the orbital respectively, Ψ₀ is a Hartree-Fock (HF) ground state, <ϕᵢϕⱼ|ϕₖϕₗ> is a two-electron integration, hₚ is a single-electron Hamiltonian operator, n is a quantity of electrons, m is an electron mass, p is a kinetic energy operator, r is a distance between q and p, and Rᵢ is a distance from an i^{th} electron to a nucleus.

In some embodiments, coulomb, commutative, and Fock operators are used. To better describe a decay tendency of long-range interaction, a coulomb operator matrix is replaced by a cubic of an element of the coulomb operator matrix. In some embodiments, in the molecular orbital basis set, a Boys-localized molecular orbital may be used to replace a canonical molecular orbital to obtain a better mobility capability of the machine learning model. In some embodiments, in an atomic orbital basis set, a symmetry-adapted atomic orbital (SAAO, |ϕ^{SAAO}>) may be used to eliminate the arbitrariness caused by the rotational covariance of a high angular momentum orbital. This application does not limit the specific form of the orbital, and other preferred orbitals may be used to optimize a subsequent calculation result.

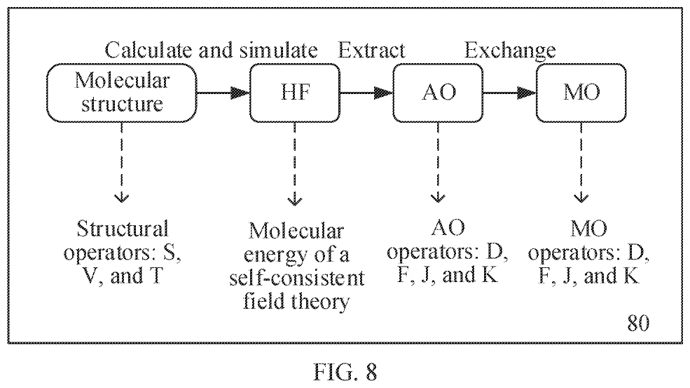

In some embodiments, there may be many different theory choices for molecular and atomic orbital generation methods. Refer to FIG. 8, which shows a block diagram of a method for obtaining operator information according to an embodiment of this application. As shown in a block diagram 80 of FIG. 8, a structural operator may be obtained directly before calculation by the self-consistent field theory (such as the HF method). Through the HF method, molecular energy of low-precision self-consistent field theory may be obtained, the atomic orbital expression form of the wave function may be extracted, and the atomic orbital may be further subjected to a matrix change to obtain the molecular orbital. The operators D, F, J, and K may be obtained based on the atomic orbital or the molecular orbital. Therefore, a molecular orbital operator or an atomic orbital operator may be obtained.

Step 340: Obtain, through a molecular energy prediction model and according to the quantum operator of the target molecule, energy information through prediction, the molecular energy prediction model including a machine learning model.

The molecular energy prediction model is a machine learning model for predicting the energy information.

The energy information is configured for representing the molecular energy obtained through prediction by the molecular energy prediction model, and this application does not limit the specific form of the energy information. In some embodiments, the energy information includes an energy difference, and the energy difference is a difference relative to the first prediction energy.

In some embodiments, an input of the molecular energy prediction model is a quantum operator of the target molecule, and an output is energy information of the target molecule.

In some embodiments, the molecular energy prediction model includes an addition kernel function based on a Gaussian process, the addition kernel function is an addition result of at least two kernel functions related to two molecules, and each kernel function is constructed based on an orbital pair in a molecule and an orbital pair in another molecule. The addition kernel function includes the at least two kernel functions.

In some embodiments, step 340 includes step 340-2 to step 340-8 (not shown in the figure).

Step 340-2: For each kernel function in the addition kernel function, obtain a first operator element from the quantum operator of the target molecule, and obtain a second operator element from a quantum operator of a sampling molecule, where the first operator element is an operator element of an orbital pair related to the kernel function in the quantum operator of the target molecule, and the second operator element is an operator element of an orbital pair related to the kernel function in the quantum operator of the sampling molecule.

In some embodiments, the kernel function is constructed based on an atomic orbital pair in a molecule and an atomic orbital pair in another molecule; or the kernel function is constructed based on a molecular orbital pair in a molecule and a molecular orbital pair in another molecule. Single-electron and two-electron quantum operators under the molecular or atomic orbital basis set construct the input end of the kernel function to construct the kernel function that represents the molecule, so that OBML provides a more accurate energy, thereby improving the precision of the entire molecular dynamics simulation, and ultimately achieving a more accurate description of the entire reaction mechanism.

In some embodiments, the kernel function is a product of at least two basic kernel functions, and different basic kernel functions are constructed based on different kernel function algorithms for a same group of orbital pairs.

Step 340-4: Obtain a calculation result of the kernel function through calculation according to the first operator element and the second operator element.

Step 340-6: Obtain a calculation result of the addition kernel function by adding the calculation result of each kernel function in the addition kernel function.

Step 340-8: Obtain the energy information according to the calculation result of the addition kernel function.

For the construction of the specific kernel function, reference is made to the following embodiment of a method for training a molecular energy prediction model, and will not be described in detail herein again. After the parameters (v, l) of the addition kernel function are determined through the method for training a molecular energy prediction model, the molecular energy prediction model can be used to predict the molecular energy.

In some embodiments, a Gaussian joint probability distribution of the target molecule X′ is given, and its mean is:

$$μ = 𝔼 [ f ( {X}^{′} ) ] = K ( {X}^{′} , X ) {\hat{K}}^{- 1} Y$$

\hat{K}=K(X, X)+σₙₒᵢₛₑ²I, where I is an identity matrix. X is a quantum operator of a sampling molecule, and a quantity of sampling molecules is at least two. For the target molecule X′ and the sampling molecule X constructing a kernel function matrix K(X′, X), the calculation of the kernel function is performed on each molecule in the target molecule X′ and each molecular in the sampling molecule X to obtain a final matrix K(X′, X). In some embodiments, each target molecule constructs the addition kernel function with each sampling molecule separately, to obtain K(X, X). A mean of the joint probability distribution is determined as the energy information.

In some embodiments, a quantity of sampling molecules is L, and L is an integer greater than 1.

In some embodiments, step 340-8 may further be determining the energy information according to the calculation result of the addition kernel function of L sampling molecules.

For any sampling molecule X of the L sampling molecules, the method of determining the calculation result of the addition kernel function of the sampling molecule is as follows:
for each kernel function in the addition kernel function, obtaining a first operator element from the quantum operator of the target molecule, and obtaining a second operator element from a quantum operator of a sampling molecule, where the first operator element is an operator element of an orbital pair related to the kernel function in the quantum operator of the target molecule, and the second operator element is an operator element of an orbital pair related to the kernel function in the quantum operator of the sampling molecule; obtaining a calculation result of the kernel function through calculation according to the first operator element and the second operator element; and obtaining a calculation result of the addition kernel function by adding the calculation result of each kernel function in the addition kernel function.

Each kernel function is constructed based on one orbital pair in one molecule and one orbital pair in another molecule. For example, one kernel function is constructed based on one orbital pair in the target molecule and one orbital pair in the sampling molecule. One orbital pair in the target molecule corresponds to an operator element; and one orbital pair in the sampling molecule corresponds to one operator element. A molecule has a plurality of electrons, and each electron occupies an orbital. It is possible that two electrons occupy the same orbital. The electron may be selected from different orbitals to calculate the quantum operator. When a kernel function is constructed, the kernel function may be constructed using the operator element corresponding to the orbital pair related to the kernel function.

In some embodiments, the kernel function is a product of at least two basic kernel functions, and different basic kernel functions are constructed based on different kernel function algorithms for a same group of orbital pairs.

For example, one kernel function is constructed based on one orbital pair in the target molecule and one orbital pair in the sampling molecule, the calculation results of a plurality of kernel functions are obtained according to the quantum operator of the target molecule and the quantum operator of the sampling molecule, and a calculation result of the addition kernel function of the sampling molecule is obtained by adding the obtained calculation result of each kernel function.

The addition kernel functions between the target molecule and the L sampling molecules are calculated respectively to obtain the calculation results of the addition kernel functions of the L sampling molecules.

In some embodiments, if a quantity of sampling molecules is L and a quantity of target molecules is 1, the target molecule needs to construct the kernel function with each sampling molecule of the L sampling molecules, and the target molecule has the calculation result of the addition kernel function with the L sampling molecules. Therefore, finally, according to the target molecule and the L sampling molecules, K(X′, X) obtained through calculation is a 1*L matrix, \hat{K}=K(X, X)+σₙₒᵢₛₑ²I. Since in this case the sampling molecule is L, \hat{K} is an L*L matrix. Y is a label value of the L sampling molecules, so Y is an L*1 matrix. The 1*L matrix K(X′, X), the L*L matrix \hat{K}, and the L*1 matrix Y are multiplied to finally obtain the energy information of the target molecule.

In some embodiments, if the quantity of sampling molecules is L, the quantity of target molecules is M, and M is a positive integer, each target molecule needs to construct a kernel function with each sampling molecule of the L sampling molecules, and each target molecule of the M target molecules has the calculation result of the addition kernel function with the L sampling molecules. Therefore, finally, according to the M target molecules and the L sampling molecules, K(X′, X) obtained through calculation is an M*L matrix, \hat{K}=K(X,X)+σₙₒᵢₛₑ²I. Since in this case the sampling molecule is L, \hat{K} is an L*L matrix. Y is a label value of the L sampling molecules, so Y is an L*1 matrix. The M*L matrix K(X′, X), the L*L matrix \hat{K}, and the L*1 matrix Y are multiplied to finally obtain an M*1 matrix, and M elements in the matrix respectively correspond to the energy information of the M target molecules.

There is no necessary relationship between the quantity L of sampling molecules and the quantity M of target molecules, and there may be any relationship between them. For example, L may be greater than M, less than M, or equal to M. For another example, L may be a multiple of M, or M may be a multiple of L.

In an example, by training a variety of different molecules simultaneously rather than training only different configurations of the same molecule, a universal molecular energy prediction model can be constructed. In another example, by training molecular energy data of different wave function theories, a molecular energy model with different wave function theories as target precision can be constructed. Such a multi-molecule universal molecular energy model can broadly predict a variety of different molecular energy under a variety of different scenarios.

The foregoing method uses a Gaussian process as a machine learning algorithm. The Gaussian process is an extremely accurate machine learning method, and generally requires little data to achieve relatively high precision compared to a neural network, which gives the user a possibility of using a small amount of data for targeted local modeling.

Step 360: Determine final prediction energy of the target molecule according to the first prediction energy and the energy information.

In some embodiments, the energy information includes an energy difference, and the energy difference is a difference relative to the first prediction energy. In some embodiments, step 360 includes step 360-2 (not shown in the figure).

Step 360-2: Determine the final prediction energy according to the energy difference and the first prediction energy.

In some embodiments, after the construction of the addition kernel function is completed, according to the Gaussian process formula, if the sampling molecules (more than 2 molecules, X is the quantum operator of the sampling molecule inputted by the training, and Y is the difference between the high-precision theory energy and the low-precision self-consistent field theory energy) (X={M^{u}}, Y=E_{diff}) can construct the foregoing addition kernel function matrix K^{add} for any target molecule X′, a mean value y of a Gaussian distribution that is equal to the energy difference Y′_{pred} of the machine learning prediction can be obtained. By adding the mean value to the molecular energy (E_{SCF}) of the low-precision self-consistent field theory, the molecular energy (E′_{high,pred}) of the high-precision theory that is predicted by the machine learning can be obtained. When the model is accurate, it is very close to the energy value (E′_{high,true}) of the real high-precision theory:

$${Y}_{pred}^{′} = μ = {K}^{add} ( {X}^{′} , X ) {- 1}^{} Y$$

$${E}_{high , pred}^{′} = {Y}_{pred}^{′} + {E}_{SCF}$$

$${E}_{high , pred}^{′} \approx {E}_{high , true}^{′}$$

In the embodiments of this application, the quantity of target molecules is not limited in this application, and the molecular energy prediction model trained in the embodiments of this application can predict the energy information of a plurality of molecules at once.

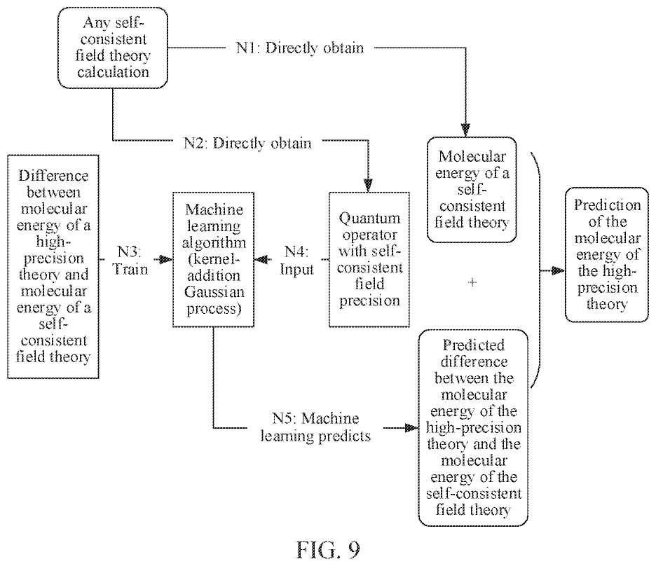

Refer to FIG. 9, which is a block diagram of a molecular energy prediction method according to an embodiment of this application. As shown in FIG. 9, the method includes steps N1 to N5.

Step N1: Directly obtain molecular energy of any self-consistent field precision.

The molecular energy of any self-consistent field precision is the first prediction energy.

Step N2: Directly obtain a quantum operator.

Step N3: Take a difference between molecular energy of a high-precision theory and molecular energy of a self-consistent field theory as a label, and train a molecular prediction model.

Step N4: Input the quantum operator to a machine learning algorithm.

That is, the quantum operator is inputted to the molecular energy prediction model.

Step N5: The machine learning predicts the difference between the molecular energy of the high-precision theory and the molecular energy of the self-consistent field theory.

That is, the energy information is determined through the molecular energy prediction model.

The molecular energy of the self-consistent field theory is added to the difference between the molecular energy of the high-precision theory and the molecular energy of the self-consistent field theory that is predicted through the machine learning to obtain the final prediction energy of the target molecule through prediction.

In some embodiments, the final prediction energy of the target molecule may be used to determine relevant information about the molecule. The relevant information may be configured for solving problems related to the molecule. In some embodiments, the final prediction energy of the target molecule is configured for determining a configuration of the target molecule; or the final prediction energy of the target molecule is configured for determining a reaction mechanism of the target molecule; or the final prediction energy of the target molecule is configured for determining a spectrum of the target molecule. The molecular energy obtained through prediction by the technical solutions provided in the embodiments of this application may be applied to any field of quantum computation that requires the molecular energy to participate in computation, and therefore, the technical solutions provided in the embodiments of this application have strong practical implications.

The technical solutions provided in the embodiments of this application may include the following beneficial effects: The first prediction energy of the target molecule and the quantum operator of the target molecule are obtained through the first calculation method (a calculation method at a lower cost). By inputting the quantum operator into the molecular energy prediction model, the energy information about the target molecule may be obtained. Through the energy information and the first prediction energy, the final prediction energy of the target molecule may be determined. The final prediction energy of the target molecule is more accurate than the first prediction energy. That is, the technical solutions provided in the embodiments of this application take the quantum operator of the molecule as an input and predicts energy of the molecule through the molecular energy prediction model. Since there are not many types of quantum operators, the types of quantum operators are basically the same between different molecules, so the molecular energy prediction model has good transferability, and the molecular energy prediction method has good universality. In addition, since the first prediction energy is obtained through a molecular energy calculation method at the lower calculation cost, the technical solutions provided in the embodiments of this application can predict the molecular energy with higher precision at the lower calculation cost.

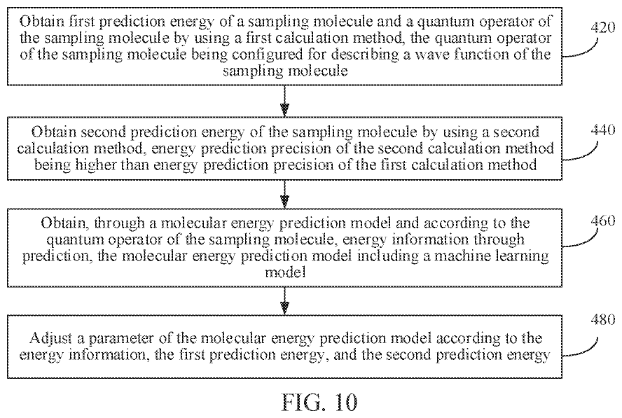

Refer to FIG. 10, which is a flowchart of a method for training a molecular energy prediction model according to an embodiment of this application. Each step of the method may be performed by the terminal device 100 in the solution implementation environment shown in FIG. 6, or by the server 200 in the solution implementation environment shown in FIG. 6. In the following method embodiment, for ease of description, a computer device is used as an execution body of each step for description. The method may include at least one step in the following steps (420 to 480):

Step 420: Obtain first prediction energy of a sampling molecule and a quantum operator of the sampling molecule by using a first calculation method, the quantum operator of the sampling molecule being configured for describing a wave function of the sampling molecule.

In some embodiments, expression forms of the quantum operator include at least one of the following: a structural operator, an atomic orbital operator, and a molecular orbital operator; the structural operator is determined based on a structure of the target molecule; the atomic orbital operator is determined based on an atomic orbital expression form of the target molecule; and the molecular orbital operator is determined based on a molecular orbital expression form of the target molecule.

In some embodiments, types of the quantum operator include at least one of the following: an overlap operator, a kinetic energy operator, a nuclear potential energy operator, a density operator, a Coulomb operator, a commutative operator, and a Fock operator.

In some embodiments, step 420 includes step 420-2 (not shown in the figure).

Step 420-2: Obtain the first prediction energy of the sampling molecule and the quantum operator of the sampling molecule by using any self-consistent field theory method.

Step 440: Obtain second prediction energy of the sampling molecule by using a second calculation method, energy prediction precision of the second calculation method being higher than energy prediction precision of the first calculation method.

In some embodiments, the first prediction energy may be considered as low-precision self-consistent field theory energy and the second prediction energy may be considered as high-precision theory energy.

The embodiments of this application do not limit the specific type of the second calculation method, and may be a wave function theory method, or other molecular energy prediction methods with higher precision than the wave function theory method.

Using the wave function theory method as an example, a near free electron approximation, a tight bound approximation, a HF method, a post-HF method, a plane wave method, an orthogonalized plane wave method, a pseudopotential method, a fixed plane wave method, and the like may be used as the second calculation method.

Using the near free electron approximation method as an example, the wave function of the near free electron approximation is a linear combination of planar wave functions.

Using the tight bound approximation as an example, in the tight bound approximation, the electron wave function is linearly superposed by the orbital wave functions of isolated atoms.

The high-precision wave function theory methods include a coupled cluster (CC) method, a Moller-plesset perturbation to second (MP2), a complete active space perturbation theory (CASPT), and the like. The foregoing method is more accurate than the self-consistent field theory method, but generally requires more calculation effort. Therefore, training is performed by using the high-precision wave function theory method to obtain a good machine learning model to predict the difference between molecular energy of the high-precision theory and the self-consistent field theory, which can be combined with the molecular energy with self-consistent field precision for high-precision molecular energy inference prediction.

The second calculation method usually has two commonly used wave functions to generate the self-consistent field theory input, one is a restricted open-shell Hartree-Fock (ROHF) method and the other is an unrestricted Hartree-Fock (UHF) method. ROHF is used in the study of open-shell systems and indicates that space parts of paired electrons are the same, but the outermost single electron occupies an open-shell orbital. The advantage is that it is a probative function of S2, but because the inner space orbital is defined to be the same, it has more variational parameters than UHF, so the energy is higher than the corresponding open-shell calculation result. UHF is used in the study of open-shell systems and indicates that the space parts of all a-spin and p-spin states are different. This is because for the open-shell systems, the outermost single electron has not only a coulombic correlation but also a commutative correlation with all electrons in the same state, but only a coulombic correlation with electrons in different states. The space parts between different spin states should be different due to the existence of an exchange correlation. The RHF method does not describe the open-shell system well because the space parts of the electrons are forced to be consistent. The two wave functions of the second calculation method are calculated using the same calculation procedure as the first calculation method, to obtain corresponding eigenvalues and eigenvectors, where the minimized eigenvector is the second prediction energy.

Step 460: Obtain, through a molecular energy prediction model and according to the quantum operator of the sampling molecule, energy information through prediction, the molecular energy prediction model including a machine learning model.

The molecular energy prediction model is a machine learning model for predicting the energy information.

In some embodiments, the molecular energy prediction model includes an addition kernel function based on a Gaussian process, the addition kernel function is an addition result of at least two kernel functions related to two molecules, and each kernel function is constructed based on an orbital pair in a molecule and an orbital pair in another molecule.

A Gaussian process can fit a non-linear function in a high-dimensional feature space, and its behavior is specified by the kernel function (covariance function). The objective of the kernel function is to describe the difference between molecules by calculating a matrix of the covariance function, in order for the Gaussian process regression model to have the property that the molecular energy can be directly predicted.

In some embodiments, the kernel function is constructed based on an atomic orbital pair in a molecule and an atomic orbital pair in another molecule; or the kernel function is constructed based on a molecular orbital pair in a molecule and a molecular orbital pair in another molecule.

In some embodiments, the kernel function is a product of at least two basic kernel functions, and different basic kernel functions are constructed based on different kernel function algorithms for a same group of orbital pairs.

In some embodiments, step 460 includes step 460-2 to step 460-8 (not shown in the figure).

Step 460-2: For each kernel function in the addition kernel function, obtain a first operator element from the quantum operator of a first sampling molecule, and obtain a second operator element from a quantum operator of a second sampling molecule, where the first operator element is an operator element of an orbital pair related to the kernel function in the quantum operator of the first sampling molecule, and the second operator element is an operator element of an orbital pair related to the kernel function in the quantum operator of the second sampling molecule.

Step 460-4: Obtain a calculation result of the kernel function through calculation according to the first operator element and the second operator element.

Step 460-6: Obtain a calculation result of the addition kernel function by adding the calculation result of each kernel function in the addition kernel function.

Step 460-8: Obtain the energy information according to the calculation result of the addition kernel function.

In some embodiments, for a determined series of operators {M^{u}}={F, J, K, S, . . . }, the addition kernel function is achieved by the following steps, where I and J represent molecules, it can be considered that I is the first sampling molecule, J is the second sampling molecule, p and q represent electrons in the molecule I, each of p and q has a respective atomic or molecular orbital, r and s represent electrons in the molecule J, and each of r and s has a respective atomic or molecular orbital. The first sampling molecule and the second sampling molecule may be the same sampling molecule or different sampling molecules.

In some embodiments, each kernel function in the addition kernel function may be at least one or more of a radial basis function kernel, a linear kernel, and a product kernel.

In some embodiments, in the first step, for the basic kernel function k between orbital pairs: construct the calculation basic kernel function k between the orbital pair (p, q) of the molecule I (referred to as Ipq subsequently) and the orbital pair (r, s) of the molecule J (referred to as Jrs subsequently), instead of directly constructing the kernel function between the molecules. In some embodiments, the molecular or atomic orbital pair Ipq and Jrs uses the radial basis function kernel (RBF) as the basic kernel function k^{RBF}:

$${k}^{RBF} ( Ipq , Jrs ) = exp ( - \frac{1}{2 {l}^{2}} {\sum }_{u} {( {M}_{Ipq}^{u} - {M}_{Jrs}^{u} )}^{2} )$$

I is a parameter of the basic kernel function, and M_{lpq}^{u} and Mⱼᵣₛ^{u} may be considered as operator elements. For example, the foregoing molecule I is a sampling molecule, the molecule J is a target molecule, M_{lpq}^{u} may be considered as a first operator element, Mⱼᵣₛ^{u} may be considered as a second operator element, and the basic kernel function k^{RBF} (Ipq,Jrs) may be considered as a calculation result of a kernel function obtained through calculation according to the first operator element and the second operator element.

Alternatively, a linear kernel may be used as the basic kernel function k^{linear}:

$${k}^{linear} ( Ipq , Jrs ) = {\sum }_{u} {( {M}_{Ipq}^{u} - {M}_{{Jrs}^{}}^{u} )}^{2}$$

In the second step, after the first step is completed, a product kernel K^{prod} of the forgoing two kernel functions is calculated to describe the long-range interaction between orbital pairs:

$${K}^{prod} ( Ipq , Jrs ) = {k}^{RBF} ( Ipq , Jrs ) {k}^{linear} ( Ipq , Jrs )$$

In the third step, a sum of the product kernel functions of all orbital pairs is then used to calculate the addition kernel function of the molecules:

$${K}^{add} ( I , J ) = {\sum }_{pqrs} {K}^{prod} ( Ipq , Jrs )$$

The long-range interaction is described by using the linear product kernel, so that the kernel function also tends to 0 at a correct speed when the long-range interaction strength tends to 0. The use of the addition kernel enables the total correlation energy of the Gaussian process regression to be decomposed to each pair of orbitals.

In some embodiments, a quantity of sampling molecules is L, where L is a positive integer greater than 1, the first sampling molecule is any one of the L sampling molecules, and the second sampling molecule is any one of the L sampling molecules.

In some embodiments, an addition kernel function may be constructed between any two sampling molecules (which may be same) of the sampling molecules, so that the calculation results of L*L addition kernel functions can be obtained.

In some embodiments, K(X, X) represents a calculation result of an addition kernel function constructed based on an input feature X. When X represents quantum operators of L sampling molecules, K(X, X) represents an L*L matrix, where a value at each position in the matrix may be considered as a calculation result of the addition kernel function of one sampling molecule and another sampling molecule.

In some embodiments, step 460-8 may further be obtaining energy information respectively corresponding to the L sampling molecules according to the calculation result of L*L addition kernel functions determined from the first sampling molecule and the second sampling molecule among the L sampling molecules.

In some embodiments, according to K(X, X) and Y, an output result for X may be determined. When X represents the quantum operators of the L sampling molecules, K(X, X) is an L*L matrix, \hat{K}=K(X, X)+σₙₒᵢₛₑ²I. Since in this case the sampling molecule is L, \hat{K} is an L*L matrix. Y is a label value of the L sampling molecules, so Y is an L*1 matrix. The L*L matrix K(X, X), the L*L matrix \hat{K}, and the L*1 matrix Y are multiplied to finally obtain an L*1 matrix, and L numbers in the matrix respectively correspond to the energy information corresponding to the L sampling molecules.

Step 480: Adjust a parameter of the molecular energy prediction model according to the first prediction information and the energy information, the first prediction energy, and the second prediction energy.

In some embodiments, the energy information includes an energy difference, and the energy difference is a difference relative to the first prediction energy.

In some embodiments, step 480 includes steps 480-2 to 480-6 (not shown in the figure).

Step 480-2: Calculate a difference between the second prediction energy and the first prediction energy, to obtain a difference result.

Step 480-4: Determine a loss function value of the molecular energy prediction model according to the difference result and the energy difference.

In some embodiments, the difference between the second prediction energy and the first prediction energy is calculated as Y, which is the label Y participating in the training, and is the difference between the molecular energy of the high-precision theory and the molecular energy of the low-precision self-consistent field theory.

In some embodiments, a sum of predicted energy differences is used as a difference degree of a difference result of the label, that is, a loss function value of the molecular energy prediction model. In some embodiments, the loss function value is a negative log marginal likelihood (−L_{θ}), and the parameter of the model is adjusted by minimizing −L_{θ}.

Step 480-6: Adjust the parameter of the molecular energy prediction model with an objective of minimizing the loss function value.

In some embodiments, the Gaussian process is a non-parameterized kernel function-based machine learning method. Assuming that the outputted label Y is a random variable (Y˜ [symbol] (μ, σ)) obeying a Gaussian distribution. For the training feature, X and its corresponding label Y, a Gaussian noise of the variance σₙₒᵢₛₑ², and a covariance function (or kernel function) K are inputted. For any input feature X′, the predicted f(X′) given is a Gaussian joint probability distribution, whose mean μ and variance σ² are:

$$μ = 𝔼 [ f ( {X}^{′} ) ] = K ( {X}^{′} , X ) {\hat{K}}^{- 1} Y$$

$${σ}^{2} = Var [ f ( {X}^{′} ) ] = K ( {X}^{′} , X ) {\hat{K}}^{- 1} K ( X , {X}^{′} )$$

\hat{K}=K(X, X)+σₙₒᵢₛₑ²I, where I is an identity matrix. The kernel function K of the Gaussian process may generally be parameterized as K_{θ}, a parameter set θ includes variance (v/Var) and lengthscale (a parameter of the kernel function, I) of the kernel function (θ={v, l}), and θ may be obtained by minimizing −L_{θ}:

$${L}_{θ} = exp ( - \frac{1}{2} {Y}^{T} {\hat{K}}^{- 1} Y - \frac{1}{2} log \vert \hat{K} \vert - \frac{N}{2} log 2 π )$$

Y^{T} represents the transpose of Y, and N represents a quantity of data participating in the training. In this application embodiment, X represents the quantum operator of the sampling molecules participating in the training, and Y represents the difference between the second prediction energy and the first prediction energy of the sampling molecule.

In some other embodiments, the parameter adjustment method may also be to preset the quantity of training times of the model, or the difference degree between output results of any two adjacent models is less than a threshold. In some embodiments, the quantity of training times of the model is preset to be 100 times, then after the 100 times training ends, it is considered that the parameter of the model has been trained. In some embodiments, the threshold is 0.01, and when the difference degree between the training result of the model and the training result of the previous model is less than 0.01, the model is considered to have been trained.

In some embodiments, the L-BFGS algorithm may be used to optimize the parameter. The specific optimization method is not limited in this application.

FIG. 9 also shows a training process of a molecular energy prediction model according to an embodiment of this application, with the following steps.

Step N1: Directly obtain molecular energy of any self-consistent field precision.

The molecular energy of any self-consistent field precision is the first prediction energy.

Step N2: Directly obtain a quantum operator.

Step N3: Take a difference between molecular energy of a high-precision theory and molecular energy of a self-consistent field theory as a label, and train a molecular prediction model.

Step N4: Input the quantum operator to a machine learning algorithm.

The molecular energy prediction model may be trained by using the quantum operator as the input feature and the difference between the molecular energy of the high-precision theory and the molecular energy of the self-consistent field theory as labels.

That is, the quantum operator with the self-consistent field theory precision is used to represent the construction of the corresponding kernel function. The difference between the high-precision theory molecular energy and the self-consistent field theory molecular energy is used as training data, and they are input into the kernel-addition Gaussian process for training, and a machine learning model that can predict the difference between the high-precision theory molecular energy and the self-consistent field theory molecular energy is finally obtained, that is, the molecular energy prediction model in this embodiment of this application.

The technical solutions provided in the embodiments of this application provide an efficient, accurate, and transferable molecular energy model construction strategy for assisting quantum chemical simulation calculation using the machine learning method. By using various quantum operators describing the properties of single electrons and double electrons and related operator operations provided by the low-precision self-consistent field method as input information, in combination with kernel-addition Gaussian process regression algorithms, the energy data of the high-precision wave function method is trained to obtain an exact and physically meaningful high-precision molecular energy prediction model. The technical solutions provided in the embodiments of this application can improve the calculation capability and precision of machine learning-based calculation quantum chemistry to a new level, at a cost significantly lower than the traditional quantum simulation.

The molecular energy prediction method and the method for training a molecular energy prediction model provided in the embodiments of this application correspond to each other, and details are not described in detail on one side, reference may be made to descriptions on the other side.

The technical solutions provided in the embodiments of this application may be deployed on a server carrying a Linux operating system or a Windows operating system and a central processing unit (CPU)/graphics processing unit (GPU) computing resource based on Python language and Cupy library. In this solution, a machine learning framework that can directly use the quantum operator obtained from the self-consistent field theory calculation as information is provided. The complexity of the algorithm of the technical solutions provided in the embodiments of this application is introduced as follows:

Table 1 specifically compares the difference in algorithmic complexity between OBML and the literature method MOB-ML in the machine learning part. Although both methods require the quantum information to construct the kernel function, that is, the calculation costs of constructing the kernel function are close, in the step of inverting the kernel function which is the bottleneck in the operation process, since each molecule has many pairs of molecular orbital combinations (for example, an organic compound with 7 heavy atoms have more than 200 molecular orbital combinations), and the quantity of Nₚₐᵢᵣ (a quantity of paired molecular orbital combinations) is much greater than that of Nₘₒₗ. For organic compounds with 7 heavy atoms, Nₚₐᵢᵣ is 200-300 and Nₘₒₗ is 1. Therefore, OBML can train a larger dataset than MOB-ML from a solution design point of view. Future further improvements under the OBML framework may enable OBML to train larger and larger datasets.

TABLE 1
Comparison of MOB-ML with OBML on machine
learning algorithm complexity

| | MOB-ML | OBML |
|---|---|---|
| Benchmark | Traditional Gaussian | Kernel-addition Gaussian |
| implementation | process | process (traditional manner) |
| Construct Kernel | O (Nₚₐᵢᵣ²), Nₚₐᵢᵣ is a | O (Nₚₐᵢᵣ²), Nₚₐᵢᵣ is a quantity of |
| Functions | quantity of paired | paired molecular/atomic orbital |
| | molecular orbital | combinations |
| | combinations | |
| Kernel function | O (Nₚₐᵢᵣ³) | O (Nₘₒₗ³), Nₘₒₗ is a quantity of |
| inversion | | molecules |

In order to verify the effectiveness of the provided solution, the technical solutions provided in the embodiments of this application are tested on a universal dataset with different theoretical and practical values: (1) multiple-reference electronic structure calculation energy prediction of a strongly-correlated system; (2) different high-precision theory calculation prediction of different free radical small molecules (open-shell system); and (3) multi-molecule universal energy model prediction on a large standard organic compound dataset.

### (1) Multiple-Reference Electronic Structure Calculation Energy Prediction of a Strongly-Correlated System

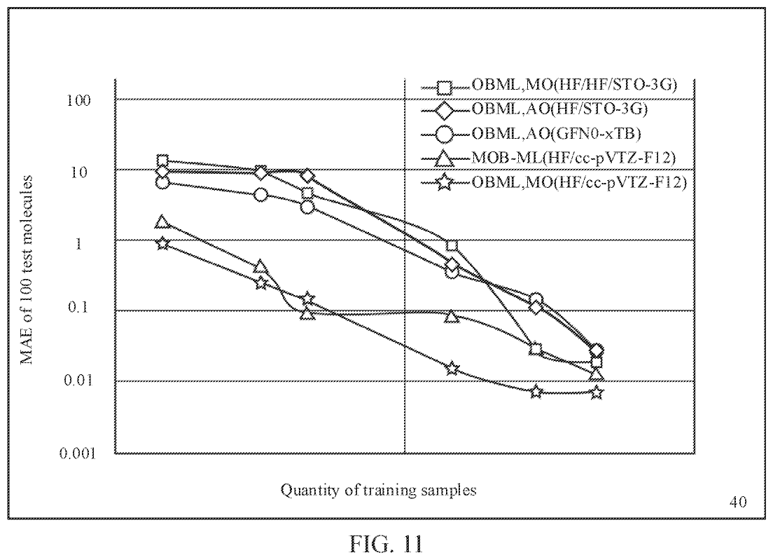

FIG. 11 shows a schematic diagram of a prediction result of an electronic structure energy, specifically a prediction result of a high-precision multi-reference electronic structure energy calculation (MRCI+Q-F12) of a traditional strong-correlated system. The precision of the model is represented by a mean absolute error (MAE), and the smaller the value, the more accurate it is. Generally, DFT cannot accurately perform the calculation of such problems. OBML represents the technical solutions provided in the embodiments of this application, MO (molecular orbital) or AO (atomic orbital) represent two common input expression forms, and HF/cc-pVTZ-F12, HF/STO-3G, and GFN0-xTB represent input ends of the self-consistent field theory at three different precision levels. The model calculation cost increases stepwise from bottom to top. The test dataset is a same one and includes 9 randomly selected H₁₀ molecule results. All different input end combinations obtain very accurate machine learning models, which illustrates the ubiquity and precision of OBML. From the lower end to the upper end of the picture, the calculation cost required at the input end increase stepwise, where there is only one set of results since MOB-ML can only accept MO input at the same basis set. Although the input of the self-consistent field theory of HF/cc-pVTZ-F12 is the most expensive, it is the most accurate theory, which is also consistent with the physical intuition. For the input mode of AO, it is more suitable to use a small basis set of the self-consistent field theory input, such as HF/STO-3G and semi-empirical GFN0-xTB. For AO, although the cost of GFN0-Xtb (0.001 s) is much less than HF/STO-3G (0.1 s), a result with similar precision may be obtained, which suggests that although GFN0-xTB is a semi-empirical theory, it can also provide input end data with sufficient physical information. In addition, although AO and MO can be converted by certain calculations, it is generally considered that the physical properties of MO are more excellent. For a same basis set and self-consistent field theory of the input end, the MO representation may obtain a slightly better result than the AO representation.

### (2) Different High-Precision Theory Calculation Prediction of Different Free Radical Small Molecules (Open-Shell System)

The calculation of the free radical molecule is also challenging for traditional quantum simulation and machine learning electronic structures, and many existing machine learning methods do not efficiently and accurately predict the molecular energy for the open-shell system. The high-precision theory calculation has two commonly used wave functions to generate the self-consistent field theory input, one is a restricted open-shell Hartree-Fock (ROHF) method and the other is an unrestricted Hartree-Fock (UHF) method. Table 2 shows the results of the use of OBML in an open-shell system. The precision is represented by MAE, the smaller the more accurate, in units of kcal/mol, compared to the results obtained with the MOB-ML method. The test datasets are all randomly selected 100 corresponding molecular configurations. Except for the Hydroxyl free radical only trains 10 molecular energy, the remaining 3 free radicals all train 80 pieces of molecular energy data.

More different input theories can be provided at the input end OBML while different wave function representations can also be used. MOB-ML can only use ROHF and the molecular orbital representation method for prediction, but OBML can use ROHF or UHF as the input theory while also using atomic and molecular orbitals for representation. In addition, at the output end, with the same training size at the same input, such as ROF/cc-pVTZ, MO and OBML can provide more accurate prediction energy as a whole than MOB-ML. For the two high-precision theories LUCCSD/cc-pVTZ and MRC+Q/cc-pVTZ, OBML gives better prediction precision on all three other free radical molecules except carbene.

TABLE 2
Different precision obtained by MOB-ML and OBML through using different kinds of
input self-consistent field theories on four different free radical molecules

| | | | Hydroxyl | Amino | Methyl | Carbene |
|---|---|---|---|---|---|---|
| MOB-ML, | ROHF/cc- | LUCCSD/cc- | 3.28E−02 | 7.80E−05 | 4.66E−04 | 7.50E−05 |
| ROHF [14] | pVTZ, MO | pVTZ | | | | |
| | | MRCI + Q/cc- | 3.36E−02 | 8.19E−05 | 8.52E−04 | 8.23E−05 |
| | | pVTZ | | | | |
| OBML, | ROHF/cc- | LUCCSD/cc- | 5.80E−05 | 4.24E−05 | 4.01E−04 | 1.54E−04 |
| ROHF | pVTZ, MO | pVTZ | | | | |
| | | MRCI + Q/cc- | 1.30E−04 | 4.38E−05 | 2.39E−04 | 1.49E−04 |
| | | pVTZ | | | | |
| | ROHF/STO- | LUCCSD/cc- | 3.15E−04 | 7.99E−04 | 1.99E−03 | 5.35E−04 |
| | 3G, MO | pVTZ | | | | |
| | | MRCI + Q/cc- | 3.64E−04 | 8.46E−04 | 1.44E−03 | 4.50E−04 |
| | | pVTZ | | | | |
| | ROHF/STO- | LUCCSD/cc- | 2.88E−02 | 5.59E−02 | 8.76E−02 | 1.86E−02 |
| | 3G, AO | pVTZ | | | | |
| | | MRCI + Q/cc- | 2.37E−02 | 9.07E−02 | 8.61E−02 | 2.01E−02 |
| | | pVTZ | | | | |
| OBML, | UHF/cc- | LUCCSD/cc- | 8.33E−05 | 5.73E−04 | 1.64E−03 | 9.92E−04 |
| UHF | pVTZ, MO | pVTZ | | | | |
| | | MRCI + Q/cc- | 8.29E−05 | 5.59E−04 | 1.57E−03 | 8.19E−04 |
| | | pVTZ | | | | |
| | UHF/STO- | LUCCSD/cc- | 3.46E−04 | 2.80E−03 | 2.21E−02 | 3.03E−03 |
| | 3G, MO | pVTZ | | | | |
| | | MRCI + Q/cc- | 3.74E−04 | 2.83E−03 | 1.54E−02 | 2.94E−03 |
| | | pVTZ | | | | |
| | UHF/STO- | LUCCSD/cc- | 8.76E−04 | 2.35E−02 | 3.07E−02 | 4.63E−03 |
| | 3G, AO | pVTZ | | | | |
| | | MRCI + Q/cc- | 9.82E−04 | 8.27E−02 | 3.36E−02 | 9.11E−03 |
| | | pVTZ | | | | |
| OBML, | xTB | LUCCSD/cc- | 5.98E−05 | 5.56E−05 | 2.86E−03 | 5.60E−03 |
| xTB | | pVTZ | | | | |
| | | MRCI + Q/cc- | 8.83E−05 | 6.65E−05 | 4.05E−03 | 5.69E−03 |
| | | pVTZ | | | | |

### (3) Multi-Molecule Universal Energy Model Prediction on a Large Standard Organic Compound Dataset

(1) and (2) are potential energy surface fittings of two single molecules. Although they are relatively challenging systems, they are still relatively simple machine learning problems. In this application scenario, it can continue to explore that the performance of OBML in the standard large dataset of organic compounds uses the datasets QM7b-T and GDB-13-T. These two standard datasets also appear in different literatures for testing. The two datasets include molecules with 7 heavy atoms and 13 heavy atoms of C, N, O, S, and Cl respectively, and the datasets include not only the optimal structure but also some thermodynamically reasonable structures. A best MOB-ML implementation would require some other high-precision theoretically calculated label information, that is, the energy corresponding to each pair of molecular orbital combination is required, not just the total molecular energy. MOB-ML can also avoid the need for much further calculation information through the kernel-addition Gaussian process and can directly predict the molecular energy.

In the technical solutions provided in the embodiments of this application, benchmark databases of a variety of applications are tested for the common scenario of ground state energy of the molecular system, and are systematically compared with other state-of-the-art machine learning solutions to illustrate advantages of the technical solutions provided in the embodiments of this application in terms of calculation time and precision.

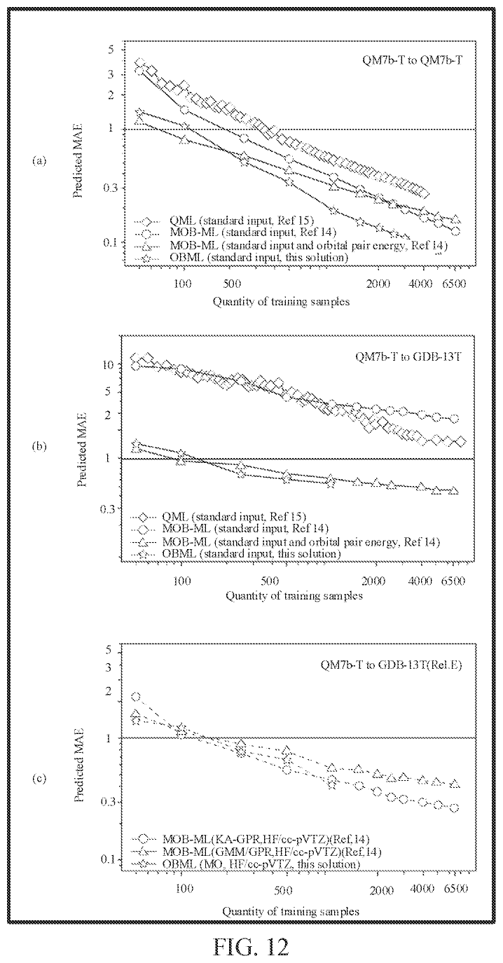

FIG. 12 shows a schematic diagram of a prediction result of a multi-molecule standardized dataset, including results of QML (Quantum Machine Learning) method, MOB-ML method, and the technical solutions (OBML) provided in the embodiments of this application. The lower the value, the closer the model prediction is to the true value, and the higher the precision of the model. It can be seen that the technical solutions provided in the embodiments of this application can provide better precision than QML and MOB-ML. FIG. 12 compares OBML with two other machine learning methods with the same calculation cost, and the model precision is evaluated using MAE. As the training data increases, all machine learning methods get better prediction precision. FIG. 12(a) shows that a model training QM7b-T molecular data predicts QM7b-T. It can be found that there is still some precision gap between the performance of OBML on the large dataset and the best MOB-ML. However, when focusing on the use of a model trained using small molecular data to predict a large molecule, it can be found that the precision difference between OBML and the best MOB-ML is relatively small and performs better than MOB-ML trained using the kernel-addition Gaussian process. This illustrates that the transferability of the small molecule model to the macromolecule of OBML is better than MOB-ML. OBML is superior to QML (MO) method as a whole in terms of precision and transferability. In addition, it can be found that the error of the relative potential energy plane on the macromolecule in FIG. 12(c) is very close to the error of the absolute energy in FIG. 12(b) for OBML and the best MOB-ML training method, but the error of MOB-ML using the kernel-addition Gaussian process is much reduced. This illustrates that the transferability loss of the kernel-addition Gaussian process based on MOB-ML is relatively high, which may be due to the lack of part of representation information. In addition, it can be found that the OBML error values in FIG. 12(b) and FIG. 12 (c) are almost close, which means that OBML meets assumptions and requirements, and the error between the obtained prediction result and the true value is almost Gaussian distributed.

Although OBML has a specific precision distance compared to the best MOB-ML implementation that requires training for energy of each pair of molecular orbital, the present result may illustrate the superior transferability of OBML and also a space for further extended improvement in the model precision. Specific solutions may include improvements in the design of the kernel function representation and improvements in the machine learning algorithm.

The following is embodiments of the apparatus of this application, which can be used to perform the embodiments of the method of this application. For details not disclosed in the embodiments of the apparatus of this application, reference is made to the embodiments of the method of this application.

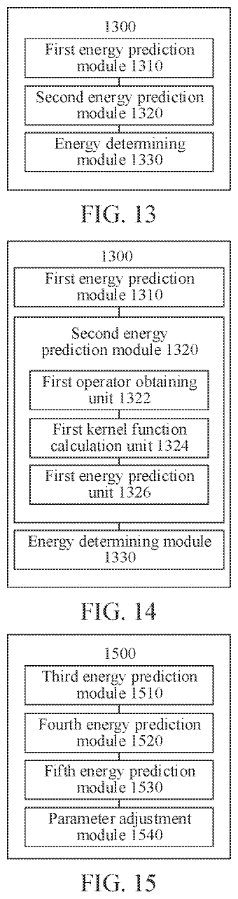

Refer to FIG. 13, which is a block diagram of a molecular energy prediction apparatus according to an embodiment of this application. The apparatus has a function of realizing the above method example, and the function may be realized by hardware or by hardware executing corresponding software. The apparatus may be the computer device described above, or may be disposed in the computer device. As shown in FIG. 13, the apparatus 1300 may include: a first energy prediction module 1310, a second energy prediction module 1320, and an energy determining module 1330.

The first energy prediction module 1310 is configured to obtain first prediction energy of a target molecule and a quantum operator of the target molecule by using a first calculation method, the quantum operator of the target molecule being configured for describing a wave function of the target molecule.

The second energy prediction module 1320 is configured to obtain, through a molecular energy prediction model and according to the quantum operator of the target molecule, energy information through prediction, the molecular energy prediction model including a machine learning model.

The energy determining module 1330 is configured to determine final prediction energy of the target molecule according to the first prediction information and the energy information.

In some embodiments, the molecular energy prediction model includes an addition kernel function based on a Gaussian process, the addition kernel function is an addition result of at least two kernel functions related to two molecules, and each kernel function is constructed based on an orbital pair in a molecule and an orbital pair in another molecule.

In some embodiments, as shown in FIG. 14, the second energy prediction module 1320 includes a first operator obtaining unit 1322, a first kernel function calculation unit 1324, and a first energy prediction unit 1326.

The first operator obtaining unit 1322 is configured to for each kernel function in the addition kernel function, obtain a first operator element from the quantum operator of the target molecule, and obtain a second operator element from a quantum operator of a sampling molecule, where the first operator element is an operator element of an orbital pair related to the kernel function in the quantum operator of the target molecule, and the second operator element is an operator element of an orbital pair related to the kernel function in the quantum operator of the sampling molecule.

The first kernel function calculation unit 1324 is configured to obtain a calculation result of the kernel function through calculation according to the first operator element and the second operator element.

The first kernel function calculation unit 1324 is further configured to obtain a calculation result of the addition kernel function by adding the calculation result of each kernel function in the addition kernel function.

The first energy prediction unit 1326 is configured to obtain the energy information according to the calculation result of the addition kernel function.

In some embodiments, a quantity of sampling molecules is L, where L is a positive integer greater than 1.

The first energy prediction unit 1326 is configured to determine the energy information according to the calculation result of the addition kernel function of L sampling molecules.

In some embodiments, the kernel function is constructed based on an atomic orbital pair in a molecule and an atomic orbital pair in another molecule; or the kernel function is constructed based on a molecular orbital pair in a molecule and a molecular orbital pair in another molecule.

In some embodiments, the kernel function is a product of at least two basic kernel functions, and different basic kernel functions are constructed based on different kernel function algorithms for a same group of orbital pairs.

In some embodiments, the energy information includes an energy difference, and the energy difference is a difference relative to the first prediction energy.

In some embodiments, the energy determining module 1330 is configured to determine the final prediction energy according to the energy difference and the first prediction energy.

In some embodiments, the first energy prediction module 1310 is configured to obtain the first prediction energy of the target molecule and the quantum operator of the target molecule by using any self-consistent field theory method.

In some embodiments, expression forms of the quantum operator include at least one of the following: a structural operator, an atomic orbital operator, and a molecular orbital operator; the structural operator is determined based on a structure of the target molecule; the atomic orbital operator is determined based on an atomic orbital expression form of the target molecule; and the molecular orbital operator is determined based on a molecular orbital expression form of the target molecule.

In some embodiments, types of the quantum operator include at least one of the following: an overlap operator, a kinetic energy operator, a nuclear potential energy operator, a density operator, a Coulomb operator, a commutative operator, and a Fock operator.

In some embodiments, the final prediction energy of the target molecule is configured for determining a configuration of the target molecule; or the final prediction energy of the target molecule is configured for determining a reaction mechanism of the target molecule; or the final prediction energy of the target molecule is configured for determining a spectrum of the target molecule.

Refer to FIG. 15, which is a block diagram of an apparatus for training a molecular energy prediction model according to an embodiment of this application. The apparatus has a function of realizing the above method example, and the function may be realized by hardware or by hardware executing corresponding software. The apparatus may be the computer device described above, or may be disposed in the computer device. As shown in FIG. 15, the apparatus 1500 may include: a third energy prediction module 1510, a fourth energy prediction module 1520, a fifth energy prediction module 1530, and a parameter adjustment module 1540.

The third energy prediction module 1510 is configured to obtain first prediction energy of a sampling molecule and a quantum operator of the sampling molecule by using a first calculation method, the quantum operator of the sampling molecule being configured for describing a wave function of the sampling molecule.

The fourth energy prediction module 1520 is configured to obtain second prediction energy of the sampling molecule by using a second calculation method, energy prediction precision of the second calculation method being higher than energy prediction precision of the first calculation method.

The fifth energy prediction module 1530 is configured to obtain, through a molecular energy prediction model and according to the quantum operator of the sampling molecule, energy information through prediction, the molecular energy prediction model including a machine learning model.

The parameter adjustment module 1540 is configured to adjust a parameter of the molecular energy prediction model according to the first prediction information and the energy information, the first prediction energy, and the second prediction energy.

In some embodiments, the molecular energy prediction model includes an addition kernel function based on a Gaussian process, the addition kernel function is an addition result of at least two kernel functions related to two molecules, and each kernel function is constructed based on an orbital pair in a molecule and an orbital pair in another molecule.

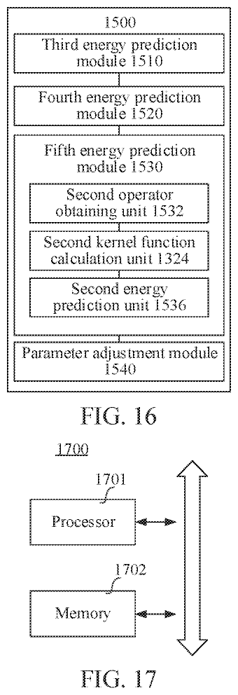

In some embodiments, as shown in FIG. 16, the fifth energy prediction module 1530 includes a second operator obtaining unit 1532, a second kernel function calculation unit 1534, and a second energy prediction unit 1536.

The second operator obtaining unit 1532 is configured to for each kernel function in the addition kernel function, obtain a first operator element from the quantum operator of a first sampling molecule, and obtain a second operator element from a quantum operator of a second sampling molecule, where the first operator element is an operator element of an orbital pair related to the kernel function in the quantum operator of the first sampling molecule, and the second operator element is an operator element of an orbital pair related to the kernel function in the quantum operator of the second sampling molecule, where the first sampling molecule and the second sampling molecule are the same or different sampling molecules.

The second kernel function calculation unit 1534 is configured to obtain a calculation result of the kernel function through calculation according to the first operator element and the second operator element.

The second kernel function calculation unit 1534 is further configured to obtain a calculation result of the addition kernel function by adding the calculation result of each kernel function in the addition kernel function.

The second energy prediction unit 1536 is configured to obtain the energy information according to the calculation result of the addition kernel function.

In some embodiments, a quantity of sampling molecules is L, the first sampling molecule is any one of the L sampling molecules, where L is a positive integer greater than 1, and the second sampling molecule is any one of the L sampling molecules.

The second energy prediction unit 1536 is configured to obtain energy information respectively corresponding to the L sampling molecules according to the calculation result of L*L addition kernel functions determined from the first sampling molecule and the second sampling molecule among the L sampling molecules.

In some embodiments, the energy information includes an energy difference, and the energy difference is a difference relative to the first prediction energy.

The parameter adjustment module 1540 is configured to calculate a difference between the second prediction energy and the first prediction energy, to obtain a difference result.

The parameter adjustment module 1540 is configured to determine a loss function value of the molecular energy prediction model according to the difference result and the energy difference.

The parameter adjustment module 1540 is configured to adjust the parameter of the molecular energy prediction model with an objective of minimizing the loss function value.

In some embodiments, the third energy prediction module 1510 is configured to obtain the first prediction energy of the sampling molecule and the quantum operator of the sampling molecule by using any self-consistent field theory method.

When the apparatus provided in the foregoing embodiments implements functions of the apparatus, the division of the foregoing functional modules is merely an example for description. In the practical application, the functions may be assigned to and completed by different functional modules according to the requirements, that is, the internal structure of the device is divided into different functional modules, to implement all or some of the functions described above. In addition, the apparatus and method embodiments provided in the foregoing embodiments belong to the same conception. For the specific implementation process, reference may be made to the method embodiments, and details are not described herein again.

FIG. 17 is a block diagram of a structure of a computer device according to an exemplary embodiment of this application.

Usually, the computer device 1700 includes: a processor 1701 and a memory 1702.

The processor 1701 may include one or more processing cores, such as a 4-core processor or a 17-core processor. The processor 1701 may be implemented by using at least one hardware form of a digital signal processing (DSP), a field programmable gate array (FPGA), and a programmable logic array (PLA). The processor 1701 may alternatively include a main processor and a coprocessor. The main processor is configured to process data in an active state, also referred to as a CPU. The coprocessor is a low-power consumption processor configured to process data in a standby state. In some embodiments, the processor 1701 may be integrated with a GPU. The GPU is configured to render and draw content that needs to be displayed on a display screen. In some embodiments, the processor 1701 may further include an artificial intelligence (AI) processor. The AI processor is configured to process a computing operation related to machine learning.

The memory 1702 may include one or more computer-readable storage media. The computer-readable storage medium may be tangible and non-transient. The memory 1702 may further include a high-speed random access memory, as well as non-volatile memory, such as one or more disk storage devices and flash storage devices. In some embodiments, the non-transitory computer-readable storage medium in the memory 1702 stores a computer program, and the computer program is loaded and executed by the processor 1701 to implement the molecular energy prediction method provided in the foregoing method embodiments or the foregoing method for training a molecular energy prediction model.

A person skilled in the art may understand that the structure shown in FIG. 17 does not constitute any limitation on the computer device 1700, and the computer device may include more components or fewer components than those shown in the figure, or some components may be combined, or a different component deployment may be used.

In an exemplary embodiment, a non-transitory computer-readable storage medium is further provided, the storage medium storing a computer program, the computer program, when executed by a processor, implementing the foregoing molecular energy prediction method or the foregoing method for training a molecular energy prediction model.

In some embodiments, the computer-readable storage medium may include: a read-only memory (ROM), a random access memory (RAM), a solid state drive (SSD), an optical disc, or the like. The RAM may include a resistance random access memory (ReRAM) and a dynamic random access memory (DRAM).

In an exemplary embodiment, a computer program product is further provided, the computer program product including a computer program, the computer program being stored in a computer-readable storage medium. A processor of a computer device reads the computer program from the computer-readable storage medium, and the processor executes the computer program, so that the computer device implements the foregoing molecular energy prediction method or the foregoing method for training a molecular energy prediction model.

It is to be understood that “plurality of” mentioned in the specification refers to two or more. “And/or” describes an association relationship between associated objects and represents that three relationships may exist. For example, A and/or B may represent the following three cases: Only A exists, both A and B exist, and only B exists. The character “/” generally represents an “or” relationship between the associated objects. In addition, the step numbers described in this specification merely schematically show a possible execution sequence of the steps. In some other embodiments, the steps may not be performed according to the number sequence. For example, two steps with different numbers may be performed simultaneously, or two steps with different numbers may be performed according to a sequence contrary to the sequence shown in the figure. This is not limited in the embodiments of this application. In this application, the term “module” in this application refers to a computer program or part of the computer program that has a predefined function and works together with other related parts to achieve a predefined goal and may be all or partially implemented by using software, hardware (e.g., processing circuitry and/or memory configured to perform the predefined functions), or a combination thereof. Each module can be implemented using one or more processors (or processors and memory). Likewise, a processor (or processors) can be used to implement one or more modules. Moreover, each module can be part of an overall module that includes the functionalities of the module.

The foregoing descriptions are merely exemplary embodiments of this application, but are not intended to limit this application. Any modification, equivalent replacement, or improvement made within the spirit and principle of this application shall fall within the protection scope of this application.

What is claimed is:

1. A molecular energy prediction method performed by a computer device, the method comprising:
obtaining first prediction energy of a target molecule and a quantum operator of the target molecule by using a first calculation method, the quantum operator of the target molecule being configured for describing a wave function of the target molecule;
predicting energy information of the target molecule through a molecular energy prediction model and according to the quantum operator of the target molecule; and
determining final prediction energy of the target molecule according to the first prediction energy and the energy information.

2. The method according to claim 1, wherein the molecular energy prediction model comprises an addition kernel function based on a Gaussian process, the addition kernel function is an addition result of at least two kernel functions related to two molecules, and each kernel function is constructed based on an orbital pair in a molecule and an orbital pair in another molecule.

3. The method according to claim 2, wherein the predicting energy information of the target molecule through a molecular energy prediction model and according to the quantum operator of the target molecule comprises:
for each kernel function in the addition kernel function, obtaining a first operator element from the quantum operator of the target molecule, and obtaining a second operator element from a quantum operator of a sampling molecule, wherein the first operator element is an operator element of an orbital pair related to the kernel function in the quantum operator of the target molecule, and the second operator element is an operator element of an orbital pair related to the kernel function in the quantum operator of the sampling molecule;
obtaining a calculation result of the kernel function through calculation according to the first operator element and the second operator element;
obtaining a calculation result of the addition kernel function by adding the calculation result of each kernel function in the addition kernel function; and
obtaining the energy information according to the calculation result of the addition kernel function.

4. The method according to claim 2, wherein a quantity of sampling molecules is L, L is a positive integer greater than 1, and the obtaining the energy information according to the calculation result of the addition kernel function comprises:
determining the energy information according to the calculation result of the addition kernel function of L sampling molecules.

5. The method according to claim 2, wherein the kernel function is a product of at least two basic kernel functions, and different basic kernel functions are constructed based on different kernel function algorithms for a same group of orbital pairs.

6. The method according to claim 1, wherein the energy information comprises an energy difference, and the energy difference is a difference relative to the first prediction energy; and
the determining final prediction energy of the target molecule according to the first prediction energy and the energy information comprises:
determining the final prediction energy according to the energy difference and the first prediction energy.

7. The method according to claim 1, wherein the obtaining first prediction energy of a target molecule and a quantum operator of the target molecule by using a first calculation method comprises:
obtaining the first prediction energy of the target molecule and the quantum operator of the target molecule by using any self-consistent field theory method.

8. The method according to claim 1, wherein expression forms of the quantum operator comprise at least one of the following: a structural operator, an atomic orbital operator, and a molecular orbital operator;
the structural operator is determined based on a structure of the target molecule;
the atomic orbital operator is determined based on an atomic orbital expression form of the target molecule; and
the molecular orbital operator is determined based on a molecular orbital expression form of the target molecule.

9. The method according to claim 1, wherein types of the quantum operator comprise at least one of the following: an overlap operator, a kinetic energy operator, a nuclear potential energy operator, a density operator, a Coulomb operator, a commutative operator, and a Fock operator.

10. A computer device, comprising a processor and a memory, the memory storing a computer program that, when executed by the processor, causes the computer device to implement a molecular energy prediction method including:
obtaining first prediction energy of a target molecule and a quantum operator of the target molecule by using a first calculation method, the quantum operator of the target molecule being configured for describing a wave function of the target molecule;
predicting energy information of the target molecule through a molecular energy prediction model and according to the quantum operator of the target molecule; and
determining final prediction energy of the target molecule according to the first prediction energy and the energy information.

11. The computer device according to claim 10, wherein the molecular energy prediction model comprises an addition kernel function based on a Gaussian process, the addition kernel function is an addition result of at least two kernel functions related to two molecules, and each kernel function is constructed based on an orbital pair in a molecule and an orbital pair in another molecule.

12. The computer device according to claim 11, wherein the predicting energy information of the target molecule through a molecular energy prediction model and according to the quantum operator of the target molecule comprises:
for each kernel function in the addition kernel function, obtaining a first operator element from the quantum operator of the target molecule, and obtaining a second operator element from a quantum operator of a sampling molecule, wherein the first operator element is an operator element of an orbital pair related to the kernel function in the quantum operator of the target molecule, and the second operator element is an operator element of an orbital pair related to the kernel function in the quantum operator of the sampling molecule;
obtaining a calculation result of the kernel function through calculation according to the first operator element and the second operator element;
obtaining a calculation result of the addition kernel function by adding the calculation result of each kernel function in the addition kernel function; and
obtaining the energy information according to the calculation result of the addition kernel function.

13. The computer device according to claim 11, wherein a quantity of sampling molecules is L, L is a positive integer greater than 1, and the obtaining the energy information according to the calculation result of the addition kernel function comprises:
determining the energy information according to the calculation result of the addition kernel function of L sampling molecules.

14. The computer device according to claim 11, wherein the kernel function is a product of at least two basic kernel functions, and different basic kernel functions are constructed based on different kernel function algorithms for a same group of orbital pairs.

15. The computer device according to claim 10, wherein the energy information comprises an energy difference, and the energy difference is a difference relative to the first prediction energy; and
the determining final prediction energy of the target molecule according to the first prediction energy and the energy information comprises:
determining the final prediction energy according to the energy difference and the first prediction energy.

16. The computer device according to claim 10, wherein the obtaining first prediction energy of a target molecule and a quantum operator of the target molecule by using a first calculation method comprises:
obtaining the first prediction energy of the target molecule and the quantum operator of the target molecule by using any self-consistent field theory method.

17. The computer device according to claim 10, wherein expression forms of the quantum operator comprise at least one of the following: a structural operator, an atomic orbital operator, and a molecular orbital operator;
the structural operator is determined based on a structure of the target molecule;
the atomic orbital operator is determined based on an atomic orbital expression form of the target molecule; and
the molecular orbital operator is determined based on a molecular orbital expression form of the target molecule.

18. The computer device according to claim 10, wherein types of the quantum operator comprise at least one of the following: an overlap operator, a kinetic energy operator, a nuclear potential energy operator, a density operator, a Coulomb operator, a commutative operator, and a Fock operator.

19. A non-transitory computer-readable storage medium storing a computer program that, when executed by a processor of a computer device, causes the computer device to implement a molecular energy prediction method including:
obtaining first prediction energy of a target molecule and a quantum operator of the target molecule by using a first calculation method, the quantum operator of the target molecule being configured for describing a wave function of the target molecule;
predicting energy information of the target molecule through a molecular energy prediction model and according to the quantum operator of the target molecule; and
determining final prediction energy of the target molecule according to the first prediction energy and the energy information.

20. The non-transitory computer-readable storage medium according to claim 19, wherein the energy information comprises an energy difference, and the energy difference is a difference relative to the first prediction energy; and
the determining final prediction energy of the target molecule according to the first prediction energy and the energy information comprises:
determining the final prediction energy according to the energy difference and the first prediction energy.
